(19) United States
(12) Patent Application Publication
(10) Pub. No.: US 2013/0101547 A1
(43) Pub. Date: Apr. 25, 2013

(54) THERMORESPONSIVE COMPOSITIONS FOR DERMATOLOGICAL USE AND METHODS THEREOF

(71) Applicant: Allergan, Inc., Irvine, CA (US)

(72) Inventors: Scott Whitcup, Laguna Hills, CA (US); Orest Olejnik, Coto de Caza, CA (US)

(73) Assignee: ALLERGAN, INC., Irvine, CA (US)

(21) Appl. No.: 13/653,271

(22) Filed: Oct. 16, 2012

Related U.S. Application Data
(60) Provisional application No. 61/549,110, filed on Oct. 19, 2011.

(51) Int. Cl. A61L 27/16; A61L 27/18; A61L 27/52

(52) U.S. Cl. CPC A61L 27/16; A61L 27/52; A61L 27/18
USPC 424/78.32; 424/78.35; 424/78.31; 424/78.38; 514/723; 424/78.37

(57) ABSTRACT

The present specification generally relates to compositions comprising a thermoresponsive polymer and methods of treating a soft tissue condition using such compositions.

### RELATED APPLICATIONS

This application claims priority to and the benefit of U.S. Provisional Patent Application No. 61/549,110, filed Oct. 19, 2011, the entire disclosure of which is incorporated here by this reference.

### BACKGROUND

Skin aging is a progressive phenomenon, occurs over time and can be affected by lifestyle factors, such as alcohol consumption, tobacco and sun exposure. Aging of the facial skin can be characterized by atrophy, slackening, and fattening. Atrophy corresponds to a massive reduction of the thickness of skin tissue. Slackening of the subcutaneous tissues leads to an excess of skin and ptosis and leads to the appearance of drooping cheeks and eye lids. Fattening refers to an increase in excess weight by swelling of the bottom of the face and neck. These changes are typically associated with dryness, loss of elasticity, and rough text

Hydrogels are cross-linked hydrophilic homopolymer or copolymer networks capable of absorbing large amounts of water or physiological fluids, while at the same time, remaining insoluble and maintaining its distinct three-dimensional structure. Its high water content and pliable properties of the polymer network render hydrogels similar to the extracellular matrix present in biological tissues. As such, hydrogels are biocompatible since its likeness to tissue minimizes inflammatory responses due to decreased tissue irritation and cell adherence, as well as facilitates cell and macromolecule integration within the polymer network. Due to this good resemblance to soft tissue, and its biocompatibility, hydrogels have been used in numerous clinical applications, such as, e.g., tissue engineering and drug delivery.

Hydrogels are composed of polymers with different architectures (block copolymers, branched polymers, multi-arm polymers like star-shaped polymers and graft polymers). Based on the polymer topology, a large variety of different assemblies can be generated and peculiar hydrogel properties, such as mesh size and/or mechanical strength can be tailored by changing the polymer design and thus by the hierarchical polymer organization. Hydrogels polymers can be natural or synthetic as well as combinations of these two types of polymers. Examples of natural polymers are polysaccharides like hyaluronic acid, alginate, dextran, chitin/chitosan, and proteins like collagen, gelatin and fibrin. Although natural polymers possess inherent biocompatibility, their use is also associated with a number of drawbacks, such as limitations in their production and purification from organisms, large batch-to-batch variation and contaminations that can possibly cause infections or immunogenicity. Moreover, their poor flexibility allows tailoring of the hydrogel properties only to a limited extent. With this respect synthetic polymers have distinct advantages. Examples of synthetic materials that have commonly been used to design hydrogels are poly(ethylene glycol) (PEG), poly(2-hydroxypropyl methacrylamide) p(HPMAm), poly(hydroxyethylmethacrylate) p(HEMA), poly(vinyl alcohol) (PVA), and poly((meth)acrylic acid) (p(M)AA).

The insoluble nature of hydrogels is due to the presence of intermolecular bonds or crosslinks between individual polymer molecules that prevents dissolution of the hydrogel as fluid is absorbed into the polymer network. Crosslinking also maintain three-dimensional structure of the polymer network. There are two primary means of polymer crosslinking seen in hydrogels. Chemical cross-linking leads to permanent covalent bonds that can be accomplished by a wide variety of chemical reactions, such as, e.g., radical polymerization, enzyme mediated polymerization, click chemistry, and Michael addition. Such reactions typically require the use of a crosslinking agent or catalyst. Physical crosslinks rely on non-permanent reversible bonds based on molecular entanglements, crystallite formation, and/or secondary forces, including, e.g., hydrophobic interactions, hydrogen bonding, stereocomplexation, inclusion complexation, or ionic interactions.

Hydrogel-based dermal fillers are useful in treating soft tissue condition and in other skin therapies because the fillers can replace lost endogenous matrix polymers, or enhance/facilitate the function of existing matrix polymers, in order to treat these skin conditions. In the past, such compositions have been used in cosmetic applications to fill wrinkles, lines, folds, scars, and to enhance dermal tissue, such as, e.g., to plump thin lips, or fill-in sunken eyes or shallow cheeks. To minimize the effect of in vivo degradation pathways as well as improve mechanical properties of the dermal filler, matrix polymers of the hydrogel are crosslinked to one another. Currently hydrogel-based dermal fillers use chemical crosslinking means to form a stabilized hydrogel.

However, problems exist using chemically crosslinked hydrogels as the bases for a dermal filler. For example, once crosslinked, the resulting hydrogel is essential similar to a solid material that cannot be introduced into an individual except by surgical means. To overcome this limitation, hydrogels are processed into microparticles in order to produce an injectable dermal filler. However, there is a fine balance between processing particles small enough to be injected into an individual with ease and comfort, but large enough to provide sufficient biomechanical and duration properties that the individual receiving the treatment will be pleased with the performance of the dermal filler. For example, the smaller the hydrogel microparticle the more fluid the dermal filler will be. Increased fluidity is desirable because it is easier to inject a fluid, as opposed to viscous, dermal filler and fluid fillers allow the use of smaller gauge needles which reduce the pain associated with the injection, and as such, increase the comfort of the individual receiving the treatment. However, the smaller the hydrogel microparticle the less mechanical strength and filler capacity there will be, and in addition, the shorter the beneficial effect of the treatment will be seen. For example, smaller hydrogel microparticles are highly soluble and susceptible to rapid degradation and, as such, are cleared rapidly when introduced into a tissue region. In addition, smaller hydrogel microparticles provide less filler volume due to their size. As such, sacrifices in performance are being made in currently produced dermal fillers in order to balance injectability and comfort with mechanical strength and filler capacity. Thus, there still remains a need for a dermal filler that provides ease and comfort, but offers a high degree of performance, such as, e.g., good mechanical strength, filler capacity, and benefit duration.

The present specification discloses dermal fillers and methods of administering dermal fillers that offer such a high degree of performance, such as, e.g., good mechanical strength, filler capacity, and benefit duration. These fillers rely on hydrogel polymers that are physically crosslinked. Because of the non-permanent character of these physical crosslinks, the polymers may be broken by a change in environmental conditions, such as, e.g., pH or temperature. As such, the disclosed dermal fillers can transition, under appropriate conditions, from a free flowing fluid of uncrosslinked polymers into a viscous crosslinked polymer network that forms a hydrogel. For example, the disclosed dermal fillers can comprise a temperature responsive polymer that is an aqueous solution at temperatures below, e.g., 25° C. but undergoes a sol-gel phase transition into a hydrogel at temperatures above, e.g., 30° C. Thus, the transition into a hydrogel near or above physiological temperatures makes the disclosed dermal fillers ideal for ‘in situ’ applications such as, e.g., treating a skin condition including, without limitation, an augmentation, a reconstruction, a disease, a disorder, a defect, or an imperfection of a body part, region or area.

### SUMMARY

The present specification provides dermal fillers useful for treating skin conditions that transition from an aqueous solution to a hydrogel under certain conditions. For example, dermal fillers comprising a thermoresponsive polymer are liquid at ambient temperatures and can undergo a sol-gel phase transition at physiological temperatures such as, e.g., upon introduction into the body. In this way, a dermal filler can be introduced into the body in a minimally invasive manner but offers a high degree of performance, such as, e.g., good mechanical strength, filler capacity, and benefit duration. Additionally, dermal fillers based on thermoresponsive polymers that rely on secondary interactions for hydrogel formation avoid potentially toxic polymerization reactions that may occur with other types of in situ polymerizing formulations, such as, e.g., photopolymerization or chemical crosslinking. Thus, thermoresponsive polymers are particularly appealing for biomedical applications as it reduces invasiveness of the treatment since the hydrogel is formed in situ upon administration.

Thus, aspects of the present specification provide a composition comprising a thermoresponsive polymer. A thermoresponsive polymer includes, without limitation, a poly(N-substituted acrylamide)-based (PNA-based) homopolymer, a PNA-based copolymer, a PNA-based block copolymer or a PNA-based interpenetrating network copolymer; a poly(vinyl ether)-based (PVE-based) homopolymer, a PVE-based copolymer, a PVE-based block copolymer or a PVE-based interpenetrating network copolymer; a poly(ethylene oxide)/poly(propylene oxide)-based (PEO/PPO-based) homopolymer, a PEO/PPO-based copolymer, a PEO/PPO-based block copolymer or a PEO/PPO-based interpenetrating network copolymer; a poly(ethylene glycol)-based (PEG-based) homopolymer, a PEG-based copolymer, a PEG-based block copolymer or a PEG-based interpenetrating network copolymer. Such compositions may further include an anti-itch agent, an anti-cellulite agent, an anti-scarring agent, an anti-inflammatory agent, an anesthetic agent, an antioxidant agent, an anti-irritant agent, a vasoconstrictor, a vasodilator, an anti-hemorrhagic agent like a hemostatic agent or anti-fibrinolytic agent, a desquamating agent, a tensioning agent, an anti-acne agent, a pigmentation agent, an anti-pigmentation agent, a moisturizing agent, or any combination thereof. In another aspect, the disclosed compositions may be used in the manufacture of a medicament.

Other aspects of the present specification provide a method of treating a soft tissue or skin condition in an individual in need thereof, the method comprising the steps of administering a composition disclosed herein to a site of the soft tissue or skin condition of the individual, wherein upon administration the composition undergoes a sol-gel phase transition to form a hydrogel, thereby improving the condition. In one aspect, the method further comprises modifying the distribution or shape of the liquid composition by external manipulation before the composition undergoes the sol-gel phase transition. Soft tissue or skin conditions treated by the disclosed compositions include, without limitation, augmentations, reconstructions, diseases, disorders, defects, or imperfections of a body part, region or area. In one aspect, a soft tissue or skin condition includes a facial augmentation, a facial reconstruction, a facial disease, a facial disorder, a facial defect, or a facial imperfection. In another aspect, a soft tissue or skin condition includes skin dehydration, a lack of skin elasticity, skin roughness, a lack of skin tautness, a skin stretch line or mark, skin paleness, a dermal divot, a sunken check, a thin lip, a retro-orbital defect, a facial fold, or a wrinkle. In another aspect, the disclosed compositions may be used to treat to a soft tissue or skin condition in an individual in need thereof.

Yet other aspects of the present specification provide a method of cosmetically enhancing a soft tissue or skin condition as disclosed herein in an individual in need thereof, the method comprising the steps of administering a composition as disclosed herein to a site of the soft tissue or skin condition of the individual, wherein upon administration the composition undergoes a sol-gel phase transition to form a hydrogel, thereby improving the condition. In one aspect, the method further comprises modifying the distribution or shape of the liquid composition by external manipulation before the composition undergoes the sol-gel phase transition. In another aspect, the disclosed compositions may be used to cosmetically enhance a soft tissue or skin condition in an individual in need thereof.

### DESCRIPTION

Stimuli-responsive polymers, also known as “smart”, “intelligent” or “environmentally sensitive” polymers, are polymers that undergo physical crosslinking upon exposure to a specific stimulus or cue resulting in formation of a hydrogel. A wide variety of stimuli or cues are known, including, without limitation, temperature, pH, electric field, pressure, or stress. Generally speaking, stimuli-responsive polymers exist in two physical states, a free flowing fluid state and a nonflowing hydrogel state, and the application or removal of a specific stimuli evokes the transition from one state to another. The transition from a solution to a hydrogel is commonly referred to as sol-gel phase transition.

One type of stimuli-responsive polymer is a thermoresponsive polymer. Thermoresponsive polymers exhibit differences in their physical state in aqueous medium in response to temperature changes. Some thermoresponsive polymers exhibit a transition from solution and hydrogel above a certain temperature. This threshold is defined as the lower critical solution temperature (LOST). Below the LOST, the polymers are soluble. Above the LOST, they become insoluble due to hydrophobic interactions between individual polymer chains, leading to hydrogel formation. In contrast, thermoresponsive polymers that exhibit a transition from solution and hydrogel below a certain temperature have this threshold defined as an upper critical solution temperature (UCST). Above the UCST, the polymers are soluble; below the UCST, the polymers become insoluble due to hydrogen bonding and physical entanglements between polymers, leading to hydrogel formation.

The compositions disclosed herein comprise a thermoresponsive that undergo a sol-to-gel transition as the temperature is raised from about room-temperature or lower to about body temperature. Thus, the compositions can be injected into an individual in a liquid or sol phase and the solution then undergo a transition to a hydrogel or gel phase, thereby forming a tissue filler, such as, e.g., a dermal filler.

Aspects of the present specification provide, in part, a composition comprising a thermoresponsive polymer. A thermoresponsive polymer may be any polymer that is biocompatible, substantially non-biodegradable and has a sol-gel phase transition temperature between about 25° C. to about 40° C. Both natural and synthetic thermoresponsive polymers may be used.

Natural thermoresponsive polymers include, e.g., proteins and polysaccharides. In aspects of this embodiment, a protein may be a collagen or a gelatin (produced by partial hydrolysis of collagen). In other aspects of this embodiment, a polysaccharide may be an agarose, a chitosan, a dextran, a xylogucan, or a cellulose derivative such as, e.g., methylcellulose (MC) and hydroxypropyl methylcellulose (HMC). Natural polymers may be used alone or in combination with synthetic polymers to design thermoresponsive polymer compositions as disclosed herein.

Natural polymers that display LOST behavior in the range between about 22° C. to about 40° C. Some cellulose derivatives (methyl and hydroxypropyl methylcellulose) at low concentrations (1-10 wt %) are liquid at low temperature, but jellify upon heating. Chemical and/or physical modification can be adopted to lower the gelation temperature, for example by addition of NaCl or decreasing the hydroxypropyl molar substitution of hydroxypropyl methylcellulose. Chitosan forms a hydrogel at about 35° C. to about 39° C. and at physiological pH when combined with glycerol phosphate disodium.

Synthetic thermoresponsive polymers include, e.g., poly(vinyl ether) (PVE) polymers, poly(N-isopropylacrylamide) (PNIPAAm) polymers, poly(N,N-diethylacrylamide) (PDEAAm) polymers, poly(N-ethylmethylacrylamide) (PNEMAAm), poly[2-(dimethylamino)ethyl methacrylate] (PDMAEMA) polymers, poly(N-vinylcaprolactum) (PVCL) polymers, poly(methyl vinyl ether) (PMVE) polymers, poly(2-ethoxyethyl vinyl ether) (PEOVE) polymers, poly(N-vinylisobytyramide (PNVIBAm) polymers, poly(N-vinyl-n-butyramide) (PNVBAm) polymers, poly(vinyl ethyl ether) (PVEE) polymers, poly(ethylene glycol) (PEG) polymers also known as poly(ethylene oxide) (PEO) polymers, derivatives thereof and copolymers thereof. PNiPAAm polymers show a LOST at about 32° C., PDEAAm polymers show a LOST at about 25° C., PNEMAAm polymers show a LOST at about 58° C., PDMAEMA polymers show a LOST at about 50° C., PVCL polymers show a LOST at about 30° C. to about 50° C., PMVE polymers show a LOST at about 34° C., PEOVE polymers show a LOST at about 20° C., PNVIBAm polymers show a LOST at about 39° C., PNVBAm polymers show a LOST at about 32° C., and PEG polymer shows a LOST at about 85° C.

Adjustment of the LOST of these thermoresponsive polymers may be achieved by the addition of side groups (derivatives) to the polymer and/or by incorporating other hydrophilic or hydrophobic monomers rendering the overall hydrophilicity of the resulting thermoresponsive copolymer higher or lower respectively. In general, the incorporation of a more hydrophilic monomer increases the LOST of a thermoresponsive polymer whereas incorporation of a more hydrophobic monomer decreases the LOST of the thermoresponsive polymer. As such, the more hydrophobic the monomer being incorporated into a thermoresponsive polymer, the lower the LOST of the resulting copolymer and vise versa.

Adjustment of the LOST of these thermoresponsive polymers may also be achieved by the addition of side groups (derivatives). For example, PEG methacrylate polymers (PEGMA), having a side-PEG chain of 2-10 ethylene oxide units (EO) present a lower LOST that varies depending on the length of the EO side chain. As another example, LOST adjustment of a thermoresponsive polymer may be ached by end-capping the polymer. In aspects of this embodiment, endcapping of the thermoresponsive polymer is with a hydroxyl group, an acetyl group, a propionyl group, a butanoyl group, a monoamino group, or a polyamino group.

The sol-gel phase transition of a synthetic thermoresponsive polymer can also be dependant on the molecular weight of the monomeric units of a polymer and/or the architecture of the polymer. The architecture of a synthetic thermoresponsive polymer as disclosed herein may be linear, branched, or star-shaped architecture.

A thermoresponsive polymer may be a homopolymer, a copolymer, a random copolymer, a grafted co-polymer, a block co-polymer, or an interpenetrating network co-polymer.

A thermoresponsive polymer disclosed herein may be a homopolymer. A thermoresponsive homopolymer refers to a polymer comprising a single monomeric species. A homopolymer may be linear, branched, or star-shaped. In an aspect of this embodiment, a thermoresponsive polymer may comprise NiPAAm monomers, and derivatives thereof, thereby producing poly(NiPAAm) or PNiPAAm homopolymers. In an aspect of this embodiment, a thermoresponsive polymer may comprise HPMAm monomers, and derivatives thereof, or derivatives thereof, such as, e.g., HPMAm-lactate (HPMAm-lac), thereby producing poly(HPMAm) or PHPMAm homopolymers and poly(HPMAm-lac) or PHPMAm-lac homopolymers, respectively.

A thermoresponsive polymer disclosed herein may be a copolymer (also known as a heteropolymer). A thermoresponsive copolymer refers to a polymer comprising two or more monomeric species. Since a copolymer comprises at least two types of monomeric units, copolymers can be classified based on how these monomers are arranged along the chain. Examples of copolymers include, without limitation, alternating copolymers, where the monomeric unit types of the copolymer regular alternating pattern; periodic copolymers, where the monomeric unit types of the copolymer are arranged in a repeating pattern; statistical copolymers, where the monomeric unit types of the copolymer are arranged based on a statistical rule; random copolymers, where the monomeric unit types of the copolymer are arranged in a random or nonstatistical manner; and block copolymers, where the copolymer comprises two or more homopolymer subunits linked by covalent bonds. Copolymers may also be described in terms of the existence of or arrangement of branches in the polymer structure. Linear copolymers comprise a single main chain whereas branched copolymers comprise a single main chain with one or more polymeric side chains. A branched copolymer as disclosed herein includes, without limitation, a simple branched copolymer, a star copolymers, a brush copolymers, and a comb copolymers.

In one embodiment, a thermoresponsive copolymer comprises PNiPAAm polymers. In aspects of this embodiment, thermoresponsive copolymers include, without limitation, P(NiPAAm-co-acrylic acid (AA)) copolymers, P(NiPAAm-co-propylacrylic acid (PAA)) copolymers, P(NiPAAm-co-HEMA) copolymers, P(NiPAAm-co-PEG-acrylate) copolymers, P(NiPAAm-co-HEMA-acrylate) copolymers, P(NiPAAm-co-N-acryloxysuccinimide (NASI)) copolymers, and P(NiPAAm-co-cysteamine) copolymers.

In another embodiment, a thermoresponsive copolymer comprises PEG polymers and an aliphatic polyester. In aspects of this embodiment, an aliphatic polyester includes, without limitation, poly(ε-caprolactone) (PCL) polymers, poly(D,L, or L-lactic acid) (PLA) polymers, poly(D,L, or L-glycolic acid) (PLA) polymers, and poly(D,L or L-lactic acid-co-glycolic acid) (PLGA) copolymers. These copolymers dissolved in water show a transition from a free flowing fluid, a sol, to a nonflowing hydrogel upon a change in temperature, which, depending on their composition, can be close to body temperature.

In yet another embodiment, a thermoresponsive copolymer comprises PEG polymers and a natural polysaccharide, such as, e.g., chitosan. In one aspect of this embodiment, a thermoresponsive copolymer comprises chitosan-co-PEG copolymers.

A thermoresponsive polymer disclosed herein may be a random copolymer. A random copolymer may be linear or branched. In one embodiment, a thermoresponsive random copolymer comprises NiPAAm monomers and pentaerythriol monostearate diacrylate (PEDAS) monomers, acrylamide (AAm) monomers, 2-hydroxyyethyl acrylate (HEA) monomers, DMAEMA monomers, or BuMA monomers. In an aspect of this embodiment, a thermoresponsive random copolymer is P(NiPAAm-co-DMAEMA-co-BuMA) copolymers.

A thermoresponsive polymer disclosed herein may be a grafted copolymer. A graft copolymer refers to a special type of branched copolymer in which the side chains are structurally distinct from the main chain. Individual side chains of a graft copolymer may be homopolymers or copolymers. In one embodiment, a thermoresponsive graft copolymer comprises poly(ethyleneimmine) (PEI) polymers grafted with PNiPAAm polymers, PEI polymers grafted with PEG polymers, chitosan grafted with PNiPAAm polymers, chitosan grafted with DMAEMA polymers, or chitosan grafted with PEI polymers.

A thermoresponsive polymer disclosed herein may be a block copolymer. A block copolymer refers to a special kind of copolymer that is made up of blocks of different polymerized monomers. The union of the homopolymer blocks may require an intermediate non-repeating subunit, known as a junction block. In one embodiment, a thermoresponsive block copolymer comprises at least one hydrophobic (thermoresponsive) polymer block and at least one hydrophilic (permanent) polymer block. Self-assembly of thermoresponsive block copolymers during the sol-gel phase transition is due to the specific combination of hydrophobic and hydrophilic polymer blocks present in the polymers.

The thermogelling properties of a thermoresponsive block copolymer are affected by the chemical structure, concentration, and hydrophobic block length. Thermoresponsive block copolymers with longer hydrophobic blocks usually undergo gelation at a lower temperature than polymers with shorter hydrophobic blocks. As such, the thermoresponsive behavior is generally viewed as a phenomenon governed by the balance of hydrophobic and hydrophilic polymer blocks in the copolymer chain and can be influenced by, e.g., the molecular weight of the polymer blocks, the composition of the polymer blocks, and the concentration of thermoresponsive block copolymer in water. Additionally, chemical and/or physical modification of the thermoresponsive block copolymer can be used to alter the temperature at which the sol-gel phase transition occurs.

Standard nomenclature useful to describe the copolymer block architecture of a thermoresponsive polymer refers to the hydrophobic block as the “A block” and the hydrophilic block as the “B block.” Blocked copolymers include polymers that comprise repeating units of A and B blocks. Non-limiting examples include, a diblock copolymer comprising AB or BA repeating units, a triblock copolymer comprising ABA, AAB, BAB, BBA, ABC, BAC, BCA as well as other combinations of repeating units, a tetrablock copolymer comprising ABAB, AABB, ABBA, BBAA, ABCA, ABCB, ABCC, ABCD, as well as other combinations of repeating units, and so on for higher number repeating unit architectures. Blocked copolymers also include multiblock copolymers. Non-limiting examples include, repeating BA or AB units to make A(BA)n or B(AB)n copolymers where n is an integer of from 2 to 8. A block copolymer may be linear or branched.

Examples of a hydrophobic A block include without limitation, a poly(α-hydroxy acid) polymer, a poly(ethylene carbonate) polymer, and bipolymers or terpolymers thereof. Non-limiting examples of a poly(α-hydroxy acid) polymers include PLA polymers, PLGA polymers, PCL polymers, poly(γ-butyrolactone) polymers, poly(ι-valerolactone) polymers, poly(ε-caprolactone co-lactic acid) (PCLA) copolymers, poly(ε-caprolactone-co-glycolic acid-co-lactic acid) (PCLGA) copolymers, poly(hydroxybutyric acid) (PHBA) polymers, poly(malic acid) (PMA) polymers.

Examples of a hydrophilic B block include without limitation, a poly(N-substituted acrylamide) polymer, a PVE polymer, and a PEO polymer. Non-limiting examples of a poly(N-substituted acrylamide) polymer include PNIPAAm polymers and PHPMAm-lac polymers.

The mean molecular weight of a hydrophobic A block and/or a hydrophilic B block may be between about 100 Da to about 20,000 Da. In aspects of this embodiment, the mean molecular weight of a hydrophobic A block and a hydrophilic B block may be between, e.g., about 200 Da and about 1,000 Da, about 200 Da and about 5,000 Da, about 200 Da and about 10,000 Da, about 300 Da and about 1,000 Da, about 300 Da and about 3,000 Da, about 300 Da and about 7,000 Da, about 500 Da and 1,500 Da, about 500 Da and 2,000 Da, about 500 Da and 3,000 Da, about 500 Da and 5,000 Da, about 1,000 Da and 1,500 Da, about 1,000 Da and 3,000 Da, or about 1,000 Da and 5,000 Da.

The concentration at which a thermoresponsive block copolymer disclosed herein remains soluble at a temperature below the sol-gel phase transition may be considered as the functional concentration. Generally speaking, thermoresponsive block copolymer concentrations of as low as 3% by weight and up to about 50% by weight can be used and still be functional. At the lower functional concentration ranges the phase transition may result in the formation of a weak hydrogel. At higher concentrations, a stronger hydrogel network is formed. In aspects of this embodiment, a concentration of a block copolymer is in the range of, e.g., about 3% to about 15%, about 3% to about 20%, about 3% to about 25%, about 3% to about 30%, about 3% to about 40%, about 5% to about 15%, about 5% to about 20%, about 5% to about 25%, about 5% to about 30%, about 5% to about 40%, about 5% to about 50%, about 10% to about 15%, about 10% to about 20%, about 10% to about 25%, about 10% to about 30%, about 10% to about 40%, or about 10% to about 50%.

The proportionate weight ratios of the hydrophobic A block to the hydrophilic B block must also be sufficient to enable the block copolymer to possess water solubility at temperatures below the sol-gel phase transition. Generally, thermoresponsive block copolymers possessing thermally reversible gelation properties are prepared when the hydrophobic A blocks comprises about 15% to about 85% by weight of the copolymer and the hydrophilic B block comprises about 15% to about 85% by weight of the copolymer.

In one embodiment, a thermoresponsive block copolymer comprises about 15% to about 50% by weight of hydrophobic A polymer block and about 50% to about 85% by weight of hydrophilic B polymer block. In an aspect of this embodiment, a thermoresponsive block copolymer comprises about 15% to about 40% by weight of hydrophobic A polymer block and about 60% to about 85% by weight of hydrophilic B polymer block. In another aspect of this embodiment, a thermoresponsive block copolymer comprises about 15% to about 35% by weight of hydrophobic A polymer block and about 65% to about 85% by weight of hydrophilic B polymer block. In yet another aspect of this embodiment, a thermoresponsive block copolymer comprises about 15% to about 30% by weight of hydrophobic A polymer block and about 70% to about 85% by weight of hydrophilic B polymer block. In still another aspect of this embodiment, a thermoresponsive block copolymer comprises about 15% to about 25% by weight of hydrophobic A polymer block and about 75% to about 85% by weight of hydrophilic B polymer block.

In one embodiment, a thermoresponsive block copolymer comprises about 20% to about 50% by weight of hydrophobic A polymer block and about 50% to about 80% by weight of hydrophilic B polymer block. In an aspect of this embodiment, a thermoresponsive block copolymer comprises about 20% to about 40% by weight of hydrophobic A polymer block and about 60% to about 80% by weight of hydrophilic B polymer block. In another aspect of this embodiment, a thermoresponsive block copolymer comprises about 25% to about 40% by weight of hydrophobic A polymer block and about 60% to about 75% by weight of hydrophilic B polymer block. In yet another aspect of this embodiment, a thermoresponsive block copolymer comprises about 20% to about 30% by weight of hydrophobic A polymer block and about 70% to about 80% by weight of hydrophilic B polymer block.

In one embodiment, a synthetic thermoresponsive polymer disclosed herein may be a triblock copolymer. In aspects of this embodiment, a triblock copolymer may be an ABA block copolymer or a BAB block copolymer. In another embodiment, a thermoresponsive triblock copolymer comprises a mean molecular weight of between about 2,000 daltons and about 20,000 daltons, and includes about 50% to about 85% by weight of hydrophobic A polymer block and about 15% to about 50% by weight of hydrophilic B polymer block.

In another embodiment, a synthetic thermoresponsive polymer disclosed herein may be a multiblock copolymer. In aspects of this embodiment, a multiblock copolymer may be an A(BA)ₙ copolymer or a B(AB)ₙ copolymer where n is an integer or from 2 to 8.

A thermoresponsive block copolymer may comprise a poly(N-substituted acrylamide)(PNA)-based block copolymer where the PNA polymer is the hydrophilic B block. The sol-gel phase transition may be elevated or reduced to a desirable value by incorporating PNA with a more hydrophilic monomer or a more hydrophobic monomer respectively. A PNA-based block copolymer disclosed herein is a substantially non-biodegradable, surface-active, block copolymer that exhibits reverse thermal gelation behavior and has a sol-gel phase transition close to body temperature. In an aspect of this embodiment, the sol-gel phase transition is between, e.g., about 25° C. to about 35° C., about 28° C. to about 35° C., about 30° C. to about 35° C., about 32° C. to about 35° C., or about 28° C. to about 32° C. A PNA-based block copolymer may be linear or branched.

In an embodiment, a thermoresponsive PNA-based block copolymer comprises a thermoresponsive PNiPAAm-based block copolymer where the PNiPAAm polymer is the hydrophilic B block. In aspects of this embodiment, a thermoresponsive PNiPAAm-based block copolymer comprises a thermoresponsive PNiPAAm-based diblock copolymer or a thermoresponsive PNiPAAm-based triblock copolymer.

In aspects of this embodiment, a thermoresponsive PNiPAAm-based diblock copolymer comprises a diblock copolymer of PNiPAAm polymers and poly(acrylic acid) (PAA) polymers or a diblock copolymer of PNiPAAm polymers and poly(propylacrylic acid) (PPAA) polymers, and combinations thereof. Non limiting examples included PNiPAAm-b-PAA diblock copolymers, PAA-b-PNiPAAm diblock copolymers, PNiPAAm-b-PPAA diblock copolymers, or PPAA-b-PNiPAAm diblock copolymers.

In other aspects of this embodiment, a thermoresponsive PNiPAAm-based diblock copolymer comprises a diblock copolymer of PNiPAAm polymers and poly(acrylamide) (PAAm) polymers, a diblock copolymer of PNiPAAm polymers and poly(2-(dimethylamino)proyl acrylamide) (PDMAPAm) polymers, a diblock copolymer of PNiPAAm polymers and poly(2-(dimethylamino)proyl acrylamide) (PDMAPAm) polymers, or a diblock copolymer of PNiPAAm polymers and poly(N,N,-dimethylacrylamide) (PDMAAm) polymers, and combinations thereof. Non limiting examples included PNiPAAm-b-PAAm diblock copolymers, PAAm-b-PNiPAAm diblock copolymers, PNiPAAm-b-PDMAPAm diblock copolymers, PDMAPAm-b-PNiPAAm diblock copolymers, PNiPAAm-b-PDMAAm diblock copolymers, or PDMAAm-b-PNiPAAm diblock copolymers.

In yet other aspects of this embodiment, a thermoresponsive PNiPAAm-based diblock copolymer comprises a diblock copolymer of PNiPAAm polymers and poly(methoxyethyl acrylate) (PMEA) polymers, a diblock copolymer of PNiPAAm polymers and poly(2-hydroxyethyl acrylate) (PHEA) polymers, a diblock copolymer of PNiPAAm polymers and poly(butyl methacrylate) (PBuMA) polymers, a diblock copolymer of PNiPAAm polymers and poly(2-hydroxyethyl methacrylate) (PHEMA) polymers, a diblock copolymer of PNiPAAm polymers and poly(methyl methacrylate) (PMMA) polymers, a diblock copolymer of PNiPAAm polymers and poly(2-(dimethylamino)ethyl methacrylate) (PDMAEMA) polymers, a diblock copolymer of PNiPAAm polymers and PDMAEMA-co-PHEMA copolymers, a diblock copolymer of PNiPAAm polymers and PEDAS polymers, and combinations thereof. Non limiting examples included PNiPAAm-b-PMEA diblock copolymers, PMEA-b-PNiPAAm diblock copolymers, PNiPAAm-b-PHEA diblock copolymers, PHEA-b-PNiPAAm diblock copolymers, PNiPAAm-b-PBuMA diblock copolymers, PBuMA-b-PNiPAAm diblock copolymers, PNiPAAm-b-PHEMA diblock copolymers, PHEMA-b-PNiPAAm diblock copolymers, PNiPAAm-b-PMMA diblock copolymers, PMMA-b-PNiPAAm diblock copolymers, PNiPAAm-b-PDMAEMA diblock copolymers, PDMAEMA-b-PNiPAAm diblock copolymers, (PDMAEMA-co-HEMA)-b-PNiPAAm diblock copolymers, PNiPAAm-b-(PDMAEMA-co-HEMA) diblock copolymers, PNiPAAm-b-PEDAS diblock copolymers, or PEDAS-b-PNiPAAm diblock copolymers.

In still other aspects of this embodiment, a thermoresponsive PNiPAAm-based diblock copolymer comprises a diblock copolymer of PNiPAAm polymers and PEG polymers. Non limiting examples included PNiPAAm-b-PEG diblock copolymers or PEG-b-PNiPAAm diblock copolymers.

In aspects of this embodiment, a thermoresponsive PNiPAAm-based triblock copolymer comprises a triblock copolymer of PNiPAAm polymers and PAA polymers or a triblock copolymer of PNiPAAm polymers and PPAA polymers, and combinations thereof. Non limiting examples included PNiPAAm-b-PAA-b PNiPAAm triblock copolymers.

In other aspects of this embodiment, a thermoresponsive PNiPAAm-based triblock copolymer comprises a triblock copolymer of PNiPAAm polymers and PAAm polymers, a triblock copolymer of PNiPAAm polymers and PDMAPAm polymers, a triblock copolymer of PNiPAAm polymers and PDMAPAm polymers, or PDMAAm polymers, and combinations thereof. Non limiting examples included PNiPAAm-b-PAAm-b-PNiPAAm triblock copolymers, PNiPAAm-b-PDMAPAm-b-PNiPAAm triblock copolymers, or PNiPAAm-b-PDMAAm-b-PNiPAAm triblock copolymers.

In yet other aspects of this embodiment, a thermoresponsive PNiPAAm-based triblock copolymer comprises a triblock copolymer of PNiPAAm polymers and PMEA polymers, a triblock copolymer of PNiPAAm polymers and PHEA polymers, a triblock copolymer of PNiPAAm polymers and PBuMA polymers, a triblock copolymer of PNiPAAm polymers and PHEMA polymers, a triblock copolymer of PNiPAAm polymers and PMMA polymers, a triblock copolymer of PNiPAAm polymers and PDMAEMA polymers, a triblock copolymer of PNiPAAm polymers and PDMAEMA-co-PHEMA copolymers, a triblock copolymer of PNiPAAm polymers and PEDAS polymers, and combinations thereof. Non limiting examples included PNiPAAm-b-PMEA-b-PNiPAAm triblock copolymers, PNiPAAm-b-PHEA-b-PNiPAAm triblock copolymers, PNiPAAm-b-PBuMA-b-PNiPAAm triblock copolymers, PNiPAAm-b-PHEMA-b-PNiPAAm triblock copolymers, PNiPAAm-b-PMMA-b-PNiPAAm triblock copolymers, PNiPAAm-b-PDMAEMA-b-PNiPAAm triblock copolymers, PNiPAAm-b-(PDMAEMA-co-HEMA)-b-PNiPAAm triblock copolymers, PNiPAAm-b-(PDMAEMA-co-HEMA)-b-PNiPAAm triblock copolymers, PNiPAAm-b-PEDAS-b-PNiPAAm triblock copolymers.

In still other aspects of this embodiment, a thermoresponsive PNiPAAm-based triblock copolymer comprises a triblock copolymer of PNiPAAm polymers and PEG polymers. Non limiting examples included PEG-b-PNiPAAm-b-PEG triblock copolymers or PnIPAAm-b-PEG-b-PNiPAAm triblock copolymers.

In other aspects of this embodiment, a thermoresponsive PNiPAAm-based triblock copolymer comprises a triblock copolymer of PNiPAAm polymers, poly(propylene oxide) (PPO) polymers, also known as poly(propylene glycol)(PPG) polymers, and poly(2-methacryloyloxyethyl phosphorylcholine (PMPC) polymers. Non limiting examples included PPO-b-PMPC-b-PNiPAAm triblock copolymers or PPO-b-PNiPAAm-b-PMPC triblock copolymers.

In another embodiment, a thermoresponsive PNA-based block copolymer comprises a thermoresponsive PDEAAm-based block copolymer where the PDEAAm polymer is the hydrophilic B block. In aspects of this embodiment, a thermoresponsive PDEAAm-based block copolymer comprises a thermoresponsive PDEAAm-based diblock copolymer or a thermoresponsive PDEAAm-based triblock copolymer.

In aspects of this embodiment, a thermoresponsive PDEAAm-based diblock copolymer comprises a diblock copolymer of PDEAAm polymers and PAA polymers or a diblock copolymer of PDEAAm polymers and PPAA polymers, and combinations thereof. Non limiting examples included PDEAAm-b-PAA diblock copolymers, PAA-b-PDEAAm diblock copolymers, PDEAAm-b-PPAA diblock copolymers, or PPAA-b-PDEAAm diblock copolymers.

In other aspects of this embodiment, a thermoresponsive PDEAAm-based diblock copolymer comprises a diblock copolymer of PDEAAm polymers and PAAm polymers, a diblock copolymer of PDEAAm polymers and PDMAPAm polymers, a diblock copolymer of PDEAAm polymers and PDMAPAm polymers, or a diblock copolymer of PDEAAm polymers and PDMAAm polymers, and combinations thereof. Non limiting examples included PDEAAm-b-PAAm diblock copolymers, PAAm-b-PDEAAm diblock copolymers, PDEAAm-b-PDMAPAm diblock copolymers, PDMAPAm-b-PDEAAm diblock copolymers, PDEAAm-b-PDMAAm diblock copolymers, or PDMAAm-b-PDEAAm diblock copolymers.

In yet other aspects of this embodiment, a thermoresponsive PDEAAm-based diblock copolymer comprises a diblock copolymer of PDEAAm polymers and PMEA polymers, a diblock copolymer of PDEAAm polymers and PHEA polymers, a diblock copolymer of PDEAAm polymers and PBuMA polymers, a diblock copolymer of PDEAAm polymers and PHEMA polymers, a diblock copolymer of PDEAAm polymers and PMMA polymers, a diblock copolymer of PDEAAm polymers and PDMAEMA polymers, a diblock copolymer of PDEAAm polymers and PDMAEMA-co-PHEMA copolymers, a diblock copolymer of PDEAAm polymers and PEDAS polymers, and combinations thereof. Non limiting examples included PDEAAm-b-PMEA diblock copolymers, PMEA-b-PDEAAm diblock copolymers, PDEAAm-b-PHEA diblock copolymers, PHEA-b-PDEAAm diblock copolymers, PDEAAm-b-PBuMA diblock copolymers, PBuMA-b-PDEAAm diblock copolymers, PDEAAm-b-PHEMA diblock copolymers, PHEMA-b-PDEAAm diblock copolymers, PDEAAm-b-PMMA diblock copolymers, PMMA-b-PDEAAm diblock copolymers, PDEAAm-b-PDMAEMA diblock copolymers, PDMAEMA-b-PDEAAm diblock copolymers, (PDMAEMA-co-HEMA)-b-PDEAAm diblock copolymers, PDEAAm-b-(PDMAEMA-co-HEMA) diblock copolymers, PDEAAm-b-PEDAS diblock copolymers, or PEDAS-b-PDEAAm diblock copolymers.

In still other aspects of this embodiment, a thermoresponsive PDEAAm-based diblock copolymer comprises a diblock copolymer of PDEAAm polymers and PEG polymers. Non limiting examples included PDEAAm-b-PEG diblock copolymers or PEG-b-PDEAAm diblock copolymers.

In aspects of this embodiment, a thermoresponsive PDEAAm-based triblock copolymer comprises a triblock copolymer of PDEAAm polymers and PAA polymers or a triblock copolymer of PDEAAm polymers and PPAA polymers, and combinations thereof. Non limiting examples included PDEAAm-b-PAA-b PDEAAm triblock copolymers.

In other aspects of this embodiment, a thermoresponsive PDEAAm-based triblock copolymer comprises a triblock copolymer of PDEAAm polymers and PAAm polymers, a triblock copolymer of PDEAAm polymers and PDMAPAm polymers, a triblock copolymer of PDEAAm polymers and PDMAPAm polymers, or PDMAAm polymers, and combinations thereof. Non limiting examples included PDEAAm-b-PAAm-b-PDEAAm triblock copolymers, PDEAAm-b-PDMAPAm-b-PDEAAm triblock copolymers, or PDEAAm-b-PDMAAm-b-PDEAAm triblock copolymers.

In yet other aspects of this embodiment, a thermoresponsive PDEAAm-based triblock copolymer comprises a triblock copolymer of PDEAAm polymers and PMEA polymers, a triblock copolymer of PDEAAm polymers and PHEA polymers, a triblock copolymer of PDEAAm polymers and PBuMA polymers, a triblock copolymer of PDEAAm polymers and PHEMA polymers, a triblock copolymer of PDEAAm polymers and PMMA polymers, a triblock copolymer of PDEAAm polymers and PDMAEMA polymers, a triblock copolymer of PDEAAm polymers and PDMAEMA-co-PHEMA copolymers, a triblock copolymer of PDEAAm polymers and PEDAS polymers, and combinations thereof. Non limiting examples included PDEAAm-b-PMEA-b-PDEAAm triblock copolymers, PDEAAm-b-PHEA-b-PDEAAm triblock copolymers, PDEAAm-b-PBuMA-b-PDEAAm triblock copolymers, PDEAAm-b-PHEMA-b-PDEAAm triblock copolymers, PDEAAm-b-PMMA-b-PDEAAm triblock copolymers, PDEAAm-b-PDMAEMA-b-PDEAAm triblock copolymers, PDEAAm-b-(PDMAEMA-co-HEMA)-b-PDEAAm triblock copolymers, PDEAAm-b-(PDMAEMA-co-HEMA)-b-PDEAAm triblock copolymers, PDEAAm-b-PEDAS-b-PDEAAm triblock copolymers.

In still other aspects of this embodiment, a thermoresponsive PDEAAm-based triblock copolymer comprises a triblock copolymer of PDEAAm polymers and PEG polymers. Non limiting examples included PEG-b-PDEAAm-b-PEG triblock copolymers or PDEAAm-b-PEG-b-PDEAAm triblock copolymers.

In yet another embodiment, a thermoresponsive PNA-based block copolymer comprises a thermoresponsive PNEMAAm-based block copolymer where the PNEMAAm polymer is the hydrophilic B block. In aspects of this embodiment, a thermoresponsive PNEMAAm-based block copolymer comprises a thermoresponsive PNEMAAm-based diblock copolymer or a thermoresponsive PNEMAAm-based triblock copolymer.

In aspects of this embodiment, a thermoresponsive PNEMAAm-based diblock copolymer comprises a diblock copolymer of PNEMAAm polymers and PAA polymers or a diblock copolymer of PNEMAAm polymers and PPAA polymers, and combinations thereof. Non limiting examples included PNEMAAm-b-PAA diblock copolymers, PAA-b-PNEMAAm diblock copolymers, PNEMAAm-b-PPAA diblock copolymers, or PPAA-b-PNEMAAm diblock copolymers.

In other aspects of this embodiment, a thermoresponsive PNEMAAm-based diblock copolymer comprises a diblock copolymer of PNEMAAm polymers and PAAm polymers, a diblock copolymer of PNEMAAm polymers and PDMAPAm polymers, a diblock copolymer of PNEMAAm polymers and PDMAPAm polymers, or a diblock copolymer of PNEMAAm polymers and PDMAAm polymers, and combinations thereof. Non limiting examples included PNEMAAm-b-PAAm diblock copolymers, PAAm-b-PNEMAAm diblock copolymers, PNEMAAm-b-PDMAPAm diblock copolymers, PDMAPAm-b-PNEMAAm diblock copolymers, PNEMAAm-b-PDMAAm diblock copolymers, or PDMAAm-b-PNEMAAm diblock copolymers.

In yet other aspects of this embodiment, a thermoresponsive PNEMAAm-based diblock copolymer comprises a diblock copolymer of PNEMAAm polymers and PMEA polymers, a diblock copolymer of PNEMAAm polymers and PHEA polymers, a diblock copolymer of PNEMAAm polymers and PBuMA polymers, a diblock copolymer of PNEMAAm polymers and PHEMA polymers, a diblock copolymer of PNEMAAm polymers and PMMA polymers, a diblock copolymer of PNEMAAm polymers and PDMAEMA polymers, a diblock copolymer of PNEMAAm polymers and PDMAEMA-co-PHEMA copolymers, a diblock copolymer of PNEMAAm polymers and PEDAS polymers, and combinations thereof. Non limiting examples included PNEMAAm-b-PMEA diblock copolymers, PMEA-b-PNEMAAm diblock copolymers, PNEMAAm-b-PHEA diblock copolymers, PHEA-b-PNEMAAm diblock copolymers, PNEMAAm-b-PBuMA diblock copolymers, PBuMA-b-PNEMAAm diblock copolymers, PNEMAAm-b-PHEMA diblock copolymers, PHEMA-b-PNEMAAm diblock copolymers, PNEMAAm-b-PMMA diblock copolymers, PMMA-b-PNEMAAm diblock copolymers, PNEMAAm-b-PDMAEMA diblock copolymers, PDMAEMA-b-PNEMAAm diblock copolymers, (PDMAEMA-co-HEMA)-b-PNEMAAm diblock copolymers, PNEMAAm-b-(PDMAEMA-co-HEMA) diblock copolymers, PNEMAAm-b-PEDAS diblock copolymers, or PEDAS-b-PNEMAAm diblock copolymers.

In still other aspects of this embodiment, a thermoresponsive PNEMAAm-based diblock copolymer comprises a diblock copolymer of PNEMAAm polymers and PEG polymers. Non limiting examples included PNEMAAm-b-PEG diblock copolymers or PEG-b-PNEMAAm diblock copolymers.

In aspects of this embodiment, a thermoresponsive PNEMAAm-based triblock copolymer comprises a triblock copolymer of PNEMAAm polymers and PAA polymers or a triblock copolymer of PNEMAAm polymers and PPAA polymers, and combinations thereof. Non limiting examples included PNEMAAm-b-PAA-b PNEMAAm triblock copolymers.

In other aspects of this embodiment, a thermoresponsive PNEMAAm-based triblock copolymer comprises a triblock copolymer of PNEMAAm polymers and PAAm polymers, a triblock copolymer of PNEMAAm polymers and PDMAPAm polymers, a triblock copolymer of PNEMAAm polymers and PDMAPAm polymers, or PDMAAm polymers, and combinations thereof. Non limiting examples included PNEMAAm-b-PAAm-b-PNEMAAm triblock copolymers, PNEMAAm-b-PDMAPAm-b-PNEMAAm triblock copolymers, or PNEMAAm-b-PDMAAm-b-PNEMAAm triblock copolymers.

In yet other aspects of this embodiment, a thermoresponsive PNEMAAm-based triblock copolymer comprises a triblock copolymer of PNEMAAm polymers and PMEA polymers, a triblock copolymer of PNEMAAm polymers and PHEA polymers, a triblock copolymer of PNEMAAm polymers and PBuMA polymers, a triblock copolymer of PNEMAAm polymers and PHEMA polymers, a triblock copolymer of PNEMAAm polymers and PMMA polymers, a triblock copolymer of PNEMAAm polymers and PDMAEMA polymers, a triblock copolymer of PNEMAAm polymers and PDMAEMA-co-PHEMA copolymers, a triblock copolymer of PNEMAAm polymers and PEDAS polymers, and combinations thereof. Non limiting examples included PNEMAAm-b-PMEA-b-PNEMAAm triblock copolymers, PNEMAAm-b-PHEA-b-PNEMAAm triblock copolymers, PNEMAAm-b-PBuMA-b-PNEMAAm triblock copolymers, PNEMAAm-b-PHEMA-b-PNEMAAm triblock copolymers, PNEMAAm-b-PMMA-b-PNEMAAm triblock copolymers, PNEMAAm-b-PDMAEMA-b-PNEMAAm triblock copolymers, PNEMAAm-b-(PDMAEMA-co-HEMA)-b-PNEMAAm triblock copolymers, PNEMAAm-b-(PDMAEMA-co-HEMA)-b-PNEMAAm triblock copolymers, PNEMAAm-b-PEDAS-b-PNEMAAm triblock copolymers.

In still other aspects of this embodiment, a thermoresponsive PNEMAAm-based triblock copolymer comprises a triblock copolymer of PNEMAAm polymers and PEG polymers. Non limiting examples included PEG-b-PNEMAAm-b-PEG triblock copolymers or PNEMAAm-b-PEG-b-PNEMAAm triblock copolymers.

A thermoresponsive block copolymer may comprise a PVE-based block copolymer where the PVE polymer is the hydrophilic B block. The sol-gel phase transition may be elevated or reduced to a desirable value by incorporating PVE with a more hydrophilic monomer or a more hydrophobic monomer respectively. A PVE-based block copolymer disclosed herein is a substantially non-biodegradable, surface-active, block copolymer that exhibits reverse thermal gelation behavior and has a sol-gel phase transition close to body temperature. In an aspect of this embodiment, the sol-gel phase transition is between, e.g., about 25° C. to about 35° C., about 28° C. to about 35° C., about 30° C. to about 35° C., about 32° C. to about 35° C., or about 28° C. to about 32° C. A PVE-based block copolymer may be linear or branched.

In an embodiment, a thermoresponsive PVE-based block copolymer comprises a thermoresponsive PVCL-based block copolymer where the PVCL polymer is the hydrophilic B block. In an aspect of this embodiment, a thermoresponsive PVCL-based block copolymer comprises a PVCL-based diblock copolymer or a PVCL-based triblock copolymer.

In another embodiment, a thermoresponsive PVE-based block copolymer comprises a thermoresponsive PMVE-based block copolymer where the PMVE polymer is the hydrophilic B block. In an aspect of this embodiment, a thermoresponsive PMVE-based block copolymer comprises a PMVE-based diblock copolymer or a PMVE-based triblock copolymer.

In yet another embodiment, a thermoresponsive PVE-based block copolymer comprises a thermoresponsive PEOVE-based block copolymer where the PEOVE polymer is the hydrophilic B block. In an aspect of this embodiment, a thermoresponsive PEOVE-based block copolymer comprises a PEOVE-based diblock copolymer or a PEOVE-based triblock copolymer. Non limiting examples include PEOVE-b-PHOVE, PEOVE-b-poly(2-(2-ethoxy)ethoxyethyl vinyl ether)(PEOEOVE)-b-PHOVE, and PEOVE-b-PEOEOVE-b-poly(2-methoxyethyl vinyl ether)(PMOVE).

In still another embodiment, a thermoresponsive PVE-based block copolymer comprises a thermoresponsive PNVIBAm-based block copolymer where the PNVIBAm polymer is the hydrophilic B block. In an aspect of this embodiment, a thermoresponsive PNVIBAm-based block copolymer comprises a PNVIBAm-based diblock copolymer or a PNVIBAm-based triblock copolymer.

In another embodiment, a thermoresponsive PVE-based block copolymer comprises a thermoresponsive PNVBAm-based block copolymer where the PNVBAm polymer is the hydrophilic B block. In an aspect of this embodiment, a thermoresponsive PNVBAm-based block copolymer comprises a PNVBAm-based diblock copolymer or a PNVBAm-based triblock copolymer.

In yet another embodiment, a thermoresponsive PVE-based block copolymer comprises a thermoresponsive PVEE-based block copolymer where the PVEE polymer is the hydrophilic B block. In an aspect of this embodiment, a thermoresponsive PVEE-based block copolymer comprises a PVEE-based diblock copolymer or a PVEE-based triblock copolymer.

In still another embodiment, a thermoresponsive PVE-based block copolymer comprises PEOEOVE-b-PMOVE and PMOVE-b-poly(octadecyl vinyl ether)(PODVE).

A thermoresponsive block copolymer may comprise a PEO-based block copolymer where the PEO is the hydrophilic B block. The sol-gel phase transition may be elevated or reduced to a desirable value by incorporating PEO with a more hydrophilic PPO monomer or a more hydrophobic PPO monomer respectively. A PEO/PPO-based block copolymer disclosed herein is a substantially non-biodegradable, surface-active, block copolymer that exhibits reverse thermal gelation behavior and has a sol-gel phase transition close to body temperature. In an aspect of this embodiment, the sol-gel phase transition is between, e.g., about 25° C. to about 35° C., about 28° C. to about 35° C., about 30° C. to about 35° C., about 32° C. to about 35° C., or about 28° C. to about 32° C. A PEO/PPO-based block copolymer may be linear or branched.

In an embodiment, a thermoresponsive PEO-based block copolymer comprises a thermoresponsive PEO/PPO-based block copolymer. In an aspect of this embodiment, a thermoresponsive PEO/PPO-based block copolymer comprises a diblock copolymer of PEO polymers and PPO polymers. Non limiting examples included PEO-b-PPO diblock polymers and PPO-b-PEO diblock polymers. In another aspect of this embodiment, a thermoresponsive PEO/PPO-based block copolymer comprises a triblock copolymer of PEO polymers and PPO polymers. Non limiting examples included PEO-b-PPO-b-PEO triblock polymers.

In yet another aspect of this embodiment, a thermoresponsive PEO/PPO-based block copolymer comprises a PEO/PPO-based tetrafunctional block copolymer where PEO and PPO polymers were condensed with ethylenediamine. In still another aspect of this embodiment, a thermoresponsive PEO/PPO-based block copolymer encapped with a monoamine and coupled to PAA polymers. Other examples of thermoresponsive PEO/PPO-based block copolymers are described in, e.g., U.S. Pat. No. 6,201,065; U.S. Pat. No. 5,252,318; U.S. Pat. No. 4,188,373, each of which is incorporated by reference in its entirety.

A thermoresponsive block copolymer may comprise a PDMAEMA-based block copolymer where the PDMAEMA polymer is the hydrophilic B block. The sol-gel phase transition may be elevated or reduced to a desirable value by incorporating PDMAEMA with a more hydrophilic monomer or a more hydrophobic monomer respectively. A PDMAEMA-based block copolymer disclosed herein is a substantially non-biodegradable, surface-active, block copolymer that exhibits reverse thermal gelation behavior and has a sol-gel phase transition close to body temperature. In an aspect of this embodiment, the sol-gel phase transition is between, e.g., about 25° C. to about 35° C., about 28° C. to about 35° C., about 30° C. to about 35° C., about 32° C. to about 35° C., or about 28° C. to about 32° C. A PDMAEMA-based block copolymer may be linear or branched.

In an embodiment, a thermoresponsive PDMAEMA-based block copolymer comprises a thermoresponsive PDMAEMA-based block copolymers. In an aspect of this embodiment, a thermoresponsive PDMAEMA-based block copolymer comprises a PDMAEMA-based diblock copolymer or a PDMAEMA-based triblock copolymer.

In yet another embodiment, a thermoresponsive block copolymer comprises a PEG-based block copolymer where the PEG is the hydrophilic B block. The sol-gel phase transition may be elevated or reduced to a desirable value by incorporating PEG with a more hydrophilic monomer or a more hydrophobic monomer respectively. A PEG-based block copolymer disclosed herein is a substantially non-biodegradable, surface-active, block copolymer that exhibits reverse thermal gelation behavior and has a sol-gel phase transition close to body temperature. In an aspect of this embodiment, the sol-gel phase transition is between, e.g., about 25° C. to about 35° C., about 28° C. to about 35° C., about 30° C. to about 35° C., about 32° C. to about 35° C., or about 28° C. to about 32° C. A PEG-based block copolymer may be linear or branched.

In an aspect of this embodiment, a thermoresponsive PEG-based block copolymer comprises a diblock copolymer of PEG polymers and polyester polymers. In aspects of this embodiment, a thermoresponsive PEG-based diblock copolymer comprises a diblock copolymer PEG polymers and PLA polymers, a diblock copolymer PEG polymers and PGA polymers, a diblock copolymer PEG polymers and PLGA polymers, and a diblock copolymer PEG polymers and PCL polymers. Non limiting examples included PEG-b-PLA diblock copolymers, PEG-b-PLA diblock copolymers, PEG-b-PGA diblock copolymers, PEG-b-PGA diblock copolymers, PEG-b-PLGA diblock copolymers, PLGA-b-PEG diblock copolymers, PEG-b-PCL diblock copolymers, and PCL-b-PEG diblock copolymers.

In another aspect of this embodiment, a thermoresponsive PEG-based block copolymer comprises a diblock copolymer of methoxy PEG (mPEG) polymers and poly(propylene fumarate (PPF) polymers. Non limiting examples included mPEG-b-PPF diblock copolymers and PPF-b-mPEG diblock copolymers.

In yet another aspect of this embodiment, a thermoresponsive PEG-based block copolymer comprises a diblock copolymer of PEG polymers and PHPMAm polymers. Non limiting examples included PEG-b-PHPMAm diblock copolymers and PHPMAm-b-PEG diblock copolymers.

In another aspect of this embodiment, a thermoresponsive PEG-based block copolymer comprises a triblock copolymer of PEG polymers and polyester polymers. In aspects of this embodiment, a thermoresponsive PEG-based triblock copolymer comprises a triblock copolymer PEG polymers and PLA polymers, a triblock copolymer PEG polymers and PGA polymers, a triblock copolymer PEG polymers and PLGA polymers, and a triblock copolymer PEG polymers and PCL polymers. Non limiting examples included PEG-b-PLA-PEG triblock copolymers, PEG-b-PGA-b-PEG triblock copolymers, PEG-b-PLGA-b-PEG triblock copolymers, PEG-b-PCL-b-PEG triblock copolymers, and PCL-b-PEG-b-PCL triblock copolymers.

In yet another aspect of this embodiment, a thermoresponsive PEG-based block copolymer comprises a triblock copolymer of methoxy PEG (mPEG) polymers and poly(propylene fumarate (PPF) polymers. Non limiting examples included mPEG-b-PPF-mPEG triblock copolymers and PPF-b-mPEG-PPF triblock copolymers.

In still another aspect of this embodiment, a thermoresponsive PEG-based block copolymer comprises a triblock copolymer of PEG polymers and PHPMAm polymers. Non limiting examples included PEG-b-PHPMAm-b-PEG triblock copolymers and PHPMAm-b-PEG-b-PHPMAm triblock copolymers.

Other examples of thermoresponsive PEG-based block copolymers are described in, e.g., U.S. Pat. No. 5,702,717; U.S. Pat. No. 5,476,909; U.S. Pat. No. 5,384,333; U.S. Pat. No. 4,716,203, each of which is incorporated by reference in its entirety.

A thermoresponsive polymer disclosed herein may be an interpenetrating network copolymer. A thermoresponsive interpenetrating network copolymer comprises two separate polymer networks that are bound together by physical entanglement as opposed to covalent bonds. One example is such an interpenetrating network is a thermoresponsive polymer comprising PAA and PAAm.

Aspects of the present specification provide, in part, a composition disclosed herein, wherein the composition does not include a therapeutic agent useful for treating an individual suffering from a disease, disorder or other aliment. A therapeutic agent as disclosed herein does not include a beneficial agent as disclosed herein, such as, e.g., an anti-itch agent, an anti-cellulite agent, an anti-scarring agent, an anti-inflammatory agent, an anesthetic agent, an anti-irritant agent, a vasoconstrictor, a vasodilator, an anti-hemorrhagic agent like a hemostatic agent or anti-fibrinolytic agent, a desquamating agent, a tensioning agent, an anti-acne agent, a pigmentation agent, an anti-pigmentation agent, or a moisturizing agent.

Aspects of the present specification provide, in part, a composition disclosed herein may further and optionally comprise a beneficial agent or combination of beneficial agents that provide a beneficial effect when the composition is administered to an individual. Such beneficial agents include, without limitation, an anti-itch agent, an anti-cellulite agent, an anti-scarring agent, an anti-inflammatory agent, an anesthetic agent, an anti-irritant agent, a vasoconstrictor, a vasodilator, an anti-hemorrhagic agent like a hemostatic agent or anti-fibrinolytic agent, a desquamating agent, a tensioning agent, an anti-acne agent, a pigmentation agent, an anti-pigmentation agent, or a moisturizing agent.

Aspects of the present specification provide, in part, a hydrogel composition disclosed herein that may optionally comprise an anesthetic agent. An anesthetic agent is preferably a local anesthetic agent, i.e., an anesthetic agent that causes a reversible local anesthesia and a loss of nociception, such as, e.g., aminoamide local anesthetics and aminoester local anesthetics. The amount of an anesthetic agent included in a composition disclosed herein is an amount effective to mitigate pain experienced by an individual upon administration of the composition. As such, the amount of an anesthetic agent included in a composition disclosed in the present specification is between about 0.1% to about 5% by weight of the total composition. Non-limiting examples of anesthetic agents include lidocaine, ambucaine, amolanone, amylocalne, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocalne, procaine, propanocaine, proparacaine, propipocaine, propoxycaine, psuedococaine, pyrrocaine, ropivacaine, salicyl alcohol, tetracaine, tolycaine, trimecaine, zolamine, combinations thereof, and salts thereof. Non-limiting examples of aminoester local anesthetics include procaine, chloroprocaine, cocaine, cyclomethycaine, cimethocaine (larocaine), propoxycaine, procaine (novocaine), proparacaine, tetracaine (amethocaine). Non-limiting examples of aminoamide local anesthetics include articaine, bupivacaine, cinchocaine (dibucaine), etidocaine, levobupivacaine, lidocaine (lignocaine), mepivacaine, piperocaine, prilocalne, ropivacaine, and trimecaine. A composition disclosed herein may comprise a single anesthetic agent or a plurality of anesthetic agents. A non-limiting example of a combination local anesthetic is lidocaine/prilocalne (EMLA).

Thus in an embodiment, a composition disclosed herein comprises an anesthetic agent and salts thereof. In aspects of this embodiment, a composition disclosed herein comprises an aminoamide local anesthetic and salts thereof or an aminoester local anesthetic and salts thereof. In other aspects of this embodiment, a composition disclosed herein comprises procaine, chloroprocaine, cocaine, cyclomethycaine, cimethocaine, propoxycaine, procaine, proparacaine, tetracaine, or salts thereof, or any combination thereof. In yet other aspects of this embodiment, a composition disclosed herein comprises articaine, bupivacaine, cinchocaine, etidocaine, levobupivacaine, lidocaine, mepivacaine, piperocaine, prilocalne, ropivacaine, trimecaine, or salts thereof, or any combination thereof. In still other aspects of this embodiment, a composition disclosed herein comprises a lidocaine/prilocalne combination.

In other aspects of this embodiment, a composition disclosed herein comprises an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an anesthetic agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an anesthetic agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anesthetic agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an anesthetic agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anesthetic agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anesthetic agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anesthetic agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an anesthetic agent.

Aspects of the present specification provide, in part, a composition disclosed herein that may optionally comprise an anti-oxidant agent. The amount of an anti-oxidant agent included in a composition disclosed herein is an amount effective to reduce or prevent degradation of a composition disclosed herein, such as, e.g., enzymatic degradation and/or chemical degradation of the composition. As such, the amount of an anti-oxidant agent included in a composition disclosed herein is between about 0.1% to about 10% by weight of the total composition. Non-limiting examples of antioxidant agents include a polyol, a flavonoid, a phytoalexin, an ascorbic acid agent, a tocopherol, a tocotrienol, a lipoic acid, a melatonin, a carotenoid, an analog or derivative thereof, and any combination thereof. A composition disclosed herein may comprise a single antioxidant agent or a plurality of antioxidant agents. Exemplary antioxidant agents are described in, e.g., US 2011/0171310, which is hereby incorporated by reference in its entirety.

A composition disclosed herein may optionally comprise a polyol. As used herein, the term “polyol” is synonymous with “sugar alcohol,” “polyhydric alcohol,” and “polyalcohol” and refers to a hydrogenated form of carbohydrate, whose carbonyl group (aldehyde or ketone, reducing sugar) has been reduced to a primary or secondary hydroxyl group (hence the alcohol), such as, e.g., mannitol from mannose, xylitol from xylose, and lactitol from lactulose. Polyols have the general formula H(HCHO)ₙ+1H. Both monosaccharides and disaccharides can form polyols; however, polyols derived from disaccharides are not entirely hydrogenated because only one aldehyde group is available for reduction. Non-limiting examples of polyols include glycerol, erythritol, threitol, arabitol, erythritol, ribitol, xylitol, galactitol (or dulcitol), gluctiol (or sorbitol), iditol, inositol, mannitol, isomalt, lactitol, maltitol, and polyglycitol. Other non-limiting examples of polyols can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

A composition disclosed herein may optionally comprise a flavonoid. A flavonoid (or bioflavonoid) refers to the class of polyphenolic ketone-containing and non-ketone-containing secondary metabolites found in plants that are well known to have diverse beneficial biochemical and antioxidant effects. Non-limiting examples of flavonoids include C-methylated flavonoids, O-methylated flavonoids, isoflavonoids, neoflavonoids, flavonolignans, furanoflavonoids, pyranoflavonoids, methylenedioxyflavonoids, prenylated flavonoids, aurones, flavones, flavonols, flavanones, flavanonols, flavan-3-ols, flavan-4-ols, leucoanthocyanidin (flavan-3,4-diols), anthocyanidins, and tannins. It is understood that these and other substances known in the art of pharmacology can be included in a composition disclosed in the present specification. See for example, Remington's Pharmaceutical Sciences Mac Publishing Company, Easton, Pa. 16^{th} Edition 1980.

A composition disclosed herein may optionally comprise an ascorbic acid agent. Ascorbic acid (Vitamin C), (5R)-[(1S)-1,2-dihydroxyethyl]-3,4-dihydroxyfuran-2(5H)-one, is a monosaccharide oxidation-reduction (redox) catalyst found in both animals and plants that reduces, and thereby neutralize, reactive oxygen species such as hydrogen peroxide. Ascorbic acid also interconverts into two unstable ketone tautomers by proton transfer, although it is the most stable in the enol form. The proton of the hydroxyl of the enol is removed. Then a pair of electrons from the resulting oxide anion pushes down to form the ketone at the 2 or 3 position and the electrons from the double bond move to the 3 or 2 position, respectively, forming the carbanion, which picks up the proton resulting in two possible forms: 1-carboxyl-2-ketone and 1-carboxyl-3-ketone. Non-limiting examples of ascorbic acid agents include ascorbic acid agents include ascorbic acid and sodium, potassium, and calcium salts of ascorbic acid, fat-soluble esters of ascorbic acid with long-chain fatty acids (ascorbyl palmitate or ascorbyl stearate), magnesium ascorbyl phosphate (MAP), sodium ascorbyl phosphate (SAP), and ascorbic acid 2-glucoside (AA2G™)

A composition disclosed herein may optionally comprise a tocopherol and/or a tocotrienol. Tocopherols and tocotrienols comprise a group of antioxidant agents collectively referred to as Vitamin E. All feature a chromanol ring, with a hydroxyl group that can donate a hydrogen atom to reduce free radicals and a hydrophobic side chain which allows for penetration into biological membranes. Both the tocopherols and tocotrienols occur in alpha, beta, gamma and delta forms, determined by the number and position of methyl groups on the chromanol ring. The tocotrienols have the same methyl structure at the ring, but differ from the analogous tocopherols by the presence of three double bonds in the hydrophobic side chain. The unsaturation of the tails gives tocotrienols only a single stereoisomeric carbon (and thus two possible isomers per structural formula, one of which occurs naturally), whereas tocopherols have 3 centers (and eight possible stereoisomers per structural formula, one of which occurs naturally). In general, the unnatural 1-isomers of tocotrienols lack almost all vitamin activity, and half of the possible 8 isomers of the tocopherols (those with 2S chirality at the ring-tail junction) also lack vitamin activity. Of the stereoisomers which retain activity, increasing methylation, especially full methylation to the alpha-form, increases vitamin activity. Non-limiting examples of Vitamin E include tocopherols (like α-tocopherol, β-tocopherol, γ-tocopherol, and δ-tocopherol), tocopherols analogs and derivatives (like tocopheryl acetate, sodium tocopheryl phosphate (STP), polyoxyethanyl-α-tocopheryl sebacate, and tocopherol polyethylene glycol 1000 succinate (TPGS)), tocotrienols (like α-tocotrienol, β-tocotrienol, γ-tocotrienol, and δ-tocotrienol), tocotrienols analogs and derivatives.

A composition disclosed herein may optionally comprise a lipoic acid (LA). Lipoic acid, (R)-5-(1,2-dithiolan-3-yl)pentanoic acid, is an organosulfur compound derived from octanoic acid that contains two vicinal sulfur atoms (at C6 and C8) attached via a disulfide bond and is thus considered to be oxidized (although either sulfur atom can exist in higher oxidation states). The carbon atom at C6 is chiral and the molecule exists as two enantiomers R-(+)-lipoic acid (RLA) and S-(−)-lipoic acid (SLA) and as a racemic mixture R/S-lipoic acid (R/S-LA). Only the R-(+)-enantiomer exists in nature and is an essential cofactor of four mitochondrial enzyme complexes.

A composition disclosed herein may optionally comprise a melatonin. Melatonin, N-acetyl-5-methoxytryptamine, is a pervasive and powerful antioxidant found in animals, plants, and microbes.

A composition disclosed herein may optionally comprise a carotenoid. There are over 600 known carotenoids; they are split into two classes, xanthophylls (which contain oxygen) and carotenes (which are purely hydrocarbons, and contain no oxygen). Non-limiting examples of carotenes include α-carotene, β-carotene, γ-carotene, δ-carotene, ε-carotene, ζ-carotene, lycopene. Non-limiting examples of xanthophylls include lutein, zeaxanthin, neoxanthin, violaxanthin, α-cryptoxanthin, and β-cryptoxanthin.

A hydrogel composition disclosed herein that may optionally comprise a Vitamin A. Vitamin A includes retinol, retinal and retinoic acid and the different geometric isomers of retinol [(2E,4E,6E,8E)-3,7-dimethyl-9-(2,6,6-trimethylcyclohex-1-enyl)nona-2,4,6,8-tetraen-1-ol], retinal and retinoic acid resulting form either a trans or cis configuration of four of the five double bonds found in the polyene chain. Non-limiting examples of Vitamin A include retinol, retinal, retinoic acid, isomers of retinol, isomers of retinal, isomers of retinoic acid, tretinoin, isotretinoin, and retinyl palmitate.

In an embodiment, a composition disclosed herein comprises an antioxidant agent in an amount sufficient to reduce or prevent degradation of a thermoresponsive polymer. In aspects of this embodiment, a composition disclosed herein comprises a polyol, a flavonoid, a phytoalexin, an ascorbic acid agent, a tocopherol, a tocotrienol, a lipoic acid, a melatonin, a carotenoid, an analog or derivative thereof, or any combination thereof.

In other aspects of this embodiment, a composition disclosed herein comprises an antioxidant agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an antioxidant agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an antioxidant agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an antioxidant agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an antioxidant agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an antioxidant agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an antioxidant agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an antioxidant agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an antioxidant agent.

Aspects of the present specification provide, in part, a composition disclosed herein that may optionally comprise a vasoconstrictor agent. The amount of a vasoconstrictor agent included in a composition disclosed herein is an amount effective to reduce, stop, and/or prevent bleeding experienced by an individual upon or after administration of the composition. Non-limiting examples of vasoconstrictor agents include α1 receptor agonists like 2-(1-naphthylmethyl)-2-imidazoline (naphazoline), (R)-4-(1-hydroxy-2-(methylamino)ethyl)benzene-1,2-diol (epinephrine), 2-amino-1-(2,5-dimethoxyphenyl)propan-1-ol (methoxamine), 4-[(1R,2S)-2-amino-1-hydroxypropyl]benzene-1,2-diol (methylnorepinephrine), 4-[(1R)-2-amino-1-hydroxyethyl]benzene-1,2-diol (norepinephrine), 3-(4,5-dihydro-1H-imidazol-2-ylmethyl)-2,4-dimethyl-6-tert-butyl-phenol (oxymetazoline), (R)-3-[-1-hydroxy-2-(methylamino)ethyl]phenol (phenylephrine or neo-synephrine), (R*,R*)-2-methylamino-1-phenylpropan-1-ol (pseudoephedrine), 4-[1-hydroxy-2-(methylamino)ethyl]phenol (synephrine or oxedrine), 2-[(2-cyclopropylphenoxy)methyl]-4,5-dihydro-1H-imidazole (cirazoline), 2-[(4-tert-butyl-2,6-dimethylphenyl)methyl]-4,5-dihydro-1H-imidazole (xylometazoline), analogs or derivatives thereof, and any combination thereof. A composition disclosed herein may comprise a single vasoconstrictor agent or a plurality of vasoconstrictor agents.

Thus in an embodiment, a composition disclosed herein comprises a vasoconstrictor agent. In aspects of this embodiment, a composition disclosed herein comprises an al receptor agonists. In aspects of this embodiment, a composition disclosed herein comprises naphazoline, epinephrine, methoxamine, methylnorepinephrine, norepinephrine, oxymetazoline, phenylephrine, pseudoephedrine, synephrine, cirazoline, xylometazoline, an analog or a derivative thereof, or any combination thereof.

In other aspects of this embodiment, a composition disclosed herein comprises a vasoconstrictor agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises a vasoconstrictor agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises a vasoconstrictor agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises a vasoconstrictor agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises a vasoconstrictor agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises a vasoconstrictor agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises a vasoconstrictor agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises a vasoconstrictor agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise a vasoconstrictor agent.

Aspects of the present specification provide, in part, a composition disclosed herein that may optionally comprise an antihemorrhagic agent. An antihemorrhagic agent includes hemostatic agents and antifibrinolytic agents. A hemostatic agent is a molecule that acts to reduce, stop, and/or prevent bleeding in the case of a ruptured blood vessel. One class of hemostatic agents is Vitamin K and its analogs or derivatives. Vitamin K and its 2-methyl-1,4-naphthoquinone derivatives is a group of lipophilic, hydrophobic vitamins that are needed for the posttranslational modification of certain proteins, mostly required for blood coagulation but also involved in metabolism pathways in bone and other tissue. The function of vitamin K in the cell is to convert glutamate in proteins to gamma-carboxyglutamate (gla). An antifibrinolytic agent is a molecule that acts to promote blood clot formation. Antifibrinolytics include aminocaproic acid (ε-aminocaproic acid) and tranexamic acid. These lysine-like drugs interfere with the formation of the fibrinolytic enzyme plasmin from its precursor plasminogen by plasminogen activators (primarily t-PA and u-PA). These drugs reversible block the lysine-binding sites of the enzymes or plasminogen and thus stop plasmin formation thereby preventing fibrinolysis and the breakdown of a blood clot. The amount of an antihemorrhagic agent included in a composition disclosed herein is an amount effective to reduce, stop, and/or prevent bleeding experienced by an individual upon or after administration of the composition. Ethamsylate (dicynene/dicynone) is another hemostatic agent. Non-limiting examples of antihemorrhagic agents include haemostatic agents like, chitosane, ethamsylate, a Vitamin K or a Vitamin K analog, such as, e.g., a Vitamin K₁ (phylloquinone, phytomenadione, or phytonadione), a Vitamin K₂ (menaquinone or menatetrenone), a Vitamin K₃ (menadione), a Vitamin K₄ (menadiol), a Vitamin K₅ (4-amino-2-methyl-1-naphthol hydrochloride), a Vitamin K₆, a Vitamin K₇, a Vitamin K_{g}, a Vitamin K₉, and a Vitamin K₁₀, antifibrinolytic agents like aminocaproic acid (ε-aminocaproic acid), tranexamic acid, serpins like aprotinin, α1-antitrypsin, C1-inhibitor, camostat, analogs or derivatives thereof, and any combination thereof. A composition disclosed herein may comprise a single antihemorrhagic agent or a plurality of antihemorrhagic agents.

Thus in an embodiment, a composition disclosed herein comprises an antihemorrhagic agent. In aspects of this embodiment, a composition disclosed herein comprises a hemostatic agent or an antifibrinolytic agent. In aspects of this embodiment, a composition disclosed herein comprises Vitamin K or a Vitamin K analog, such as, e.g., a Vitamin K₁, a Vitamin K₂, a Vitamin K₃, a Vitamin K₄, a Vitamin K₅, a Vitamin K₆, a Vitamin K₇, a Vitamin K_{g}, a Vitamin K₉, and a Vitamin K₁₀, ε-aminocaproic acid, tranexamic acid, serpins like aprotinin, α1-antitrypsin, C1-inhibitor, camostat, an analog or a derivative thereof, or any combination thereof.

In other aspects of this embodiment, a composition disclosed herein comprises antihemorrhagic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises antihemorrhagic agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises antihemorrhagic agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises antihemorrhagic agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises antihemorrhagic agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises antihemorrhagic agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises antihemorrhagic agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises antihemorrhagic agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise antihemorrhagic agent.

Aspects of the present specification provide, in part, a composition disclosed herein that may optionally comprise an anti-itch agent. The amount of an anti-itch agent included in a composition disclosed herein is an amount effective to mitigate an itch response experienced by an individual upon administration of the composition. Non-limiting examples of anti-itch agents include methyl sulphonyl methane, sodium bicarbonate, calamine, allantoin, kaolin, peppermint, tea tree oil, camphor, menthol, hydrocortisone, analogs or derivatives thereof, and any combination thereof. A composition disclosed herein may comprise a single anti-itch agent or a plurality of anti-itch agents.

Thus in an embodiment, a composition disclosed herein comprises an anti-itch agent. In aspects of this embodiment, a composition disclosed herein comprises methyl sulphonyl methane, sodium bicarbonate, calamine, allantoin, kaolin, peppermint, tea tree oil, camphor, menthol, hydrocortisone, an analog or derivative thereof, or any combination thereof.

In other aspects of this embodiment, a composition disclosed herein comprises an anti-itch agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an anti-itch agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an anti-itch agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anti-itch agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an anti-itch agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anti-itch agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anti-itch agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anti-itch agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an anti-itch agent.

Aspects of the present specification provide, in part, a composition disclosed herein that may optionally comprise an anti-cellulite agent. The amount of an anti-cellulite agent included in a composition disclosed herein is an amount effective to mitigate a fatty deposit experienced by an individual upon administration of the composition. Non-limiting examples of anti-cellulite agents include forskolin, xanthine compounds such as, but not limited to, caffeine, theophylline, theobromine, and aminophylline, analogs or derivatives thereof, and any combination thereof. A composition disclosed herein may comprise a single anti-cellulite agent or a plurality of anti-cellulite agents.

Thus in an embodiment, a composition disclosed herein comprises an anti-cellulite agent. In aspects of this embodiment, a composition disclosed herein comprises forskolin, a xanthine compound, an analog or derivative thereof, or any combination thereof.

In other aspects of this embodiment, a composition disclosed herein comprises an anti-cellulite agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an anti-cellulite agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an anti-cellulite agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anti-cellulite agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an anti-cellulite agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anti-cellulite agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anti-cellulite agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anti-cellulite agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an anti-cellulite agent.

Aspects of the present specification provide, in part, a composition disclosed herein that may optionally comprise an anti-scarring agent. The amount of an anti-scarring agent included in a composition disclosed herein is an amount effective to mitigate a scaring response experienced by an individual upon administration of the composition. Non-limiting examples of anti-scarring agents include IFN-γ, fluorouracil, poly(lactic-co-glycolic acid), methylated polyethylene glycol, polylactic acid, polyethylene glycol, analogs or derivatives thereof, and any combination thereof. A composition disclosed herein may comprise a single anti-scarring agent or a plurality of anti-scarring agents.

Thus in an embodiment, a composition disclosed herein comprises an anti-scarring agent. In aspects of this embodiment, a composition disclosed herein comprises IFN-γ, fluorouracil, poly(lactic-co-glycolic acid), methylated polyethylene glycol, polylactic acid, polyethylene glycol, an analog or derivative thereof, or any combination thereof.

In other aspects of this embodiment, a composition disclosed herein comprises an anti-scarring agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an anti-scarring agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an anti-scarring agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anti-scarring agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an anti-scarring agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anti-scarring agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anti-scarring agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anti-scarring agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an anti-scarring agent.

Aspects of the present specification provide, in part, a composition disclosed herein that may optionally comprise an anti-inflammatory agent. The amount of an anti-inflammatory agent included in a composition disclosed herein is an amount effective to mitigate an inflammatory and/or irritating response experienced by an individual upon administration of the composition. Non-limiting examples of anti-inflammatory agents include dexamethasone, prednisolone, corticosterone, budesonide, estrogen, sulfasalazine, mesalamine, cetirizine, diphenhydramine, antipyrine, methyl salicylate, loratadine, thymol (2-isopropyl-5-methylphenol), carvacrol (5-isopropyl-2-methylphenol), bisabolol (6-Methyl-2-(4-methylcyclohex-3-enyl)hept-5-en-2-ol), allantoin, eucalyptol, phenazone (antipyrin), propyphenazone, and Non-steroidal anti-inflammatory drugs (NSAIDs) include, without limitation, propionic acid derivatives like ibuprofen, naproxen, fenoprofen, ketoprofen, flurbiprofen, and oxaprozin; acetic acid derivatives like indomethacin, sulindac, etodolac, ketorolac, diclofenac, and nabumetone; enolic acid (oxicam) derivatives like piroxicam, meloxicam, tenoxicam, droxicam, lornoxicam, isoxicam; fenamic acid derivatives like mefenamic acid, meclofenamic acid, flufenamic acid, and tolfenamic acid; and selective COX-2 inhibitors (coxibs) like celecoxib, rofecoxib, valdecoxib, parecoxib, lumiracoxib, etoricoxib, and firocoxib, analogs or derivatives thereof, and any combination thereof. A composition disclosed herein may comprise a single anti-inflammatory agent or a plurality of anti-inflammatory agents.

Thus in an embodiment, a composition disclosed herein comprises an anti-inflammatory agent. In aspects of this embodiment, a composition disclosed herein comprises dexamethasone, prednisolone, corticosterone, budesonide, estrogen, sulfasalazine, mesalamine, cetirizine, diphenhydramine, antipyrine, methyl salicylate, loratadine, thymol (2-isopropyl-5-methylphenol), carvacrol (5-isopropyl-2-methylphenol), bisabolol (6-Methyl-2-(4-methylcyclohex-3-enyl)hept-5-en-2-ol), allantoin, eucalyptol, phenazone (antipyrin), propyphenazone, a NSAID, an analog or derivative thereof, or any combination thereof.

In other aspects of this embodiment, a composition disclosed herein comprises an anti-inflammatory agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In yet other aspects, a composition disclosed herein comprises an anti-inflammatory agent in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In still other aspects, a composition disclosed herein comprises an anti-inflammatory agent in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anti-inflammatory agent in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises an anti-inflammatory agent at a concentration of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anti-inflammatory agent at a concentration of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anti-inflammatory agent at a concentration of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anti-inflammatory agent at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

In another embodiment, a composition disclosed herein does not comprise an anesthetic agent.

A composition disclosed herein is in a sol phase at room temperature or below. In aspects of this embodiment, a composition is in its sol phase at, e.g., about 30° C. or lower, about 28° C. or lower, about 25° C. or lower, about 22° C. or lower, about 20° C. or lower, about 18° C. or lower, or about 16° C. or lower.

A composition disclosed herein is injectable. As used herein, the term “injectable” refers to a material having the properties necessary to administer the composition into a skin region of an individual using an injection device with a fine needle. As used herein, the term “fine needle” refers to a needle that is 22 gauge or smaller. Injectability of a composition disclosed herein is a property of a thermoresponsive polymer as disclosed herein when it is in its sol phase.

In aspect of this embodiment, a composition disclosed herein is injectable through a fine needle. In other aspects of this embodiment, a composition disclosed herein is injectable through a needle of, e.g., about 22 gauge, about 27 gauge, about 30 gauge, or about 32 gauge. In yet other aspects of this embodiment, a composition disclosed herein is injectable through a needle of, e.g., 22 gauge or smaller, 27 gauge or smaller, 30 gauge or smaller, or 32 gauge or smaller. In still other aspects of this embodiment, a composition disclosed herein is injectable through a needle of, e.g., about 22 gauge to about 32 gauge, about 22 gauge to about 27 gauge, or about 27 gauge to about 32 gauge.

In aspects of this embodiment, a composition disclosed herein can be injected with an extrusion force of about 60 N, about 55 N, about 50 N, about 45 N, about 40 N, about 35 N, about 30 N, about 25 N, about 20 N, or about 15 N. In other aspects of this embodiment, a composition disclosed herein can be injected through a 27 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less. In yet other aspects of this embodiment, a composition disclosed herein can be injected through a 30 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less. In still other aspects of this embodiment, a composition disclosed herein can be injected through a 32 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less.

A composition disclosed herein exhibits cohesivity. Cohesivity, also referred to as cohesion cohesive attraction, cohesive force, or compression force is a physical property of a material, caused by the intermolecular attraction between like-molecules within the material that acts to unite the molecules. Cohesivity is expressed in terms of grams-force (gmf). Cohesiveness is affected by, among other factors, the molecular weight ratio of the polymer, the degree of polymer crosslinking, and the pH of the composition. A composition should be sufficiently cohesive as to remain localized to a site of administration. Additionally, in certain applications, a sufficient cohesiveness is important for a composition to retain its shape, and thus functionality, in the event of mechanical load cycling. As such, in one embodiment, a composition disclosed herein exhibits cohesivity, on par with water. In yet another embodiment, a hydrogel composition disclosed herein exhibits sufficient cohesivity to remain localized to a site of administration. In still another embodiment, a composition disclosed herein exhibits sufficient cohesivity to retain its shape. In a further embodiment, a hydrogel composition disclosed herein exhibits sufficient cohesivity to retain its shape and functionality.

In aspects of this embodiment, a composition disclosed herein has a cohesivity of, e.g., about 10 gmf, about 20 gmf, about 30 gmf, about 40 gmf, about 50 gmf, about 60 gmf, about 70 gmf, about 80 gmf, about 90 gmf, about 100 gmf, about 150 gmf, or about 200 gmf. In other aspects of this embodiment, a hydrogel composition disclosed herein has a cohesivity of, e.g., at least 10 gmf, at least 20 gmf, at least 30 gmf, at least 40 gmf, at least 50 gmf, at least 60 gmf, at least 70 gmf, at least 80 gmf, at least 90 gmf, at least 100 gmf, at least 150 gmf, or at least 200 gmf. In yet other aspects of this embodiment, a composition disclosed herein has a cohesivity of, e.g., at most 10 gmf, at most 20 gmf, at most 30 gmf, at most 40 gmf, at most 50 gmf, at most 60 gmf, at most 70 gmf, at most 80 gmf, at most 90 gmf, at most 100 gmf, at most 150 gmf, or at most 200 gmf. In yet other aspects of this embodiment, a composition disclosed herein has a cohesivity of, e.g., about 50 gmf to about 150 gmf, about 60 gmf to about 140 gmf, about 70 gmf to about 130 gmf, about 80 gmf to about 120 gmf, or about 90 gmf to about 110 gmf.

In yet other aspects of this embodiment, a composition disclosed herein has a cohesivity of, e.g., about 10 gmf to about 50 gmf, about 25 gmf to about 75 gmf, about 50 gmf to about 150 gmf, about 100 gmf to about 200 gmf, about 100 gmf to about 300 gmf, about 100 gmf to about 400 gmf, about 100 gmf to about 500 gmf, about 200 gmf to about 300 gmf, about 200 gmf to about 400 gmf, about 200 gmf to about 500 gmf, about 200 gmf to about 600 gmf, about 200 gmf to about 700 gmf, about 300 gmf to about 400 gmf, about 300 gmf to about 500 gmf, about 300 gmf to about 600 gmf, about 300 gmf to about 700 gmf, about 300 gmf to about 800 gmf, about 400 gmf to about 500, about 400 gmf to about 600, about 400 gmf to about 700, about 400 gmf to about 800, about 500 gmf to about 600 gmf, about 500 gmf to about 700 gmf, about 500 gmf to about 800 gmf, about 600 gmf to about 700 gmf, about 600 gmf to about 800 gmf, about 700 gmf to about 800 gmf, about 1000 gmf to about 2000 gmf, about 1000 gmf to about 3000 gmf, or about 2000 gmf to about 3000 gmf.

A composition disclosed herein exhibits substantial stability. As used herein, the term “stability” or “stable” when referring to a composition disclosed herein refers to a composition that is not prone to degrading, decomposing, or breaking down to any substantial or significant degree while stored before administration to an individual. Non-limiting examples of substantial stability include less than 10% degradation of a composition over a time period measured, less than 5% degradation of a composition over a time period measured, less than 3% degradation of a composition over a time period measured, less than 1% degradation of a composition over a time period measured. As used herein, the term “substantial heat stability”, “substantially heat stable”, “autoclave stable”, or “steam sterilization stable” refers to a composition disclosed herein that is substantially stable when subjected to a heat treatment as disclosed herein.

Stability of a composition disclosed herein can be determined by subjecting a composition to a heat treatment, such as, e.g., steam sterilization at normal pressure or under pressure (e.g., autoclaving). Preferably the heat treatment is carried out at a temperature of at least about 100° C. for between about one minute and about 10 minutes. Substantial stability of a composition disclosed herein can be evaluated 1) by assessing the clarity and color of a composition after sterilization with a clear and uncolored composition being indicative of a substantially stable composition; 2) by determining the change in the extrusion force (EF) of a composition disclosed herein after sterilization, where the change in extrusion force less 2N is indicative of a substantially stable composition as measured by (the extrusion force of a composition with the specified additives) minus (the extrusion force of the a composition without the added additives); 3) by determining the change in rheological properties of a composition disclosed herein after sterilization, where the change in tan δ1 Hz of less than 0.1 is indicative of a substantially stable composition as measured by (tan δ1 Hz of gel formulation with additives) minus (tan δ1 Hz of gel formulation without additives) and/or 4) assessing the thermoresponsive behavior of the polymer. As such, a substantially stable composition disclosed herein retains one or more of the following characteristics after sterilization: clarity (transparency and translucency), homogeneousness, extrusion force, cohesiveness, polymer concentration, agent(s) concentration, osmolarity, pH, or other rheological characteristics desired by the hydrogel before the heat treatment.

Long term stability of a composition disclosed herein can be determined by subjecting a composition to a heat treatment, such as, e.g., storage in an about 45° C. environment for about 32 days. Stability of a composition after such a heat treatment is equivalent to about 1 year to 3 years at room temperature. Long term stability of a composition disclosed herein can be evaluated 1) by assessing the clarity and color of a composition after the 45° C. heat treatment, with a clear and uncolored composition being indicative of a substantially stable hydrogel composition; 2) by determining the change in the extrusion force (ΔF) of a composition disclosed herein after the 45° C. heat treatment, where the change in extrusion force less 2N is indicative of a substantially stable composition as measured by (the extrusion force of a hydrogel composition with the specified additives before the 45° C. heat treatment) minus (the extrusion force of the a composition with the specified additives after the 45° C. heat treatment); 3) by determining the change in rheological properties of a composition disclosed herein after sterilization, where the change in tan δ1 Hz of less than 0.1 is indicative of a substantially stable composition as measured by (tan δ1 Hz of gel formulation with the specified additives before the 45° C. heat treatment) minus (tan δ1 Hz of gel formulation with the specified additives after the 45° C. heat treatment) and/or 4) assessing the thermoresponsive behavior of the polymer. As such, a long term stability of a composition disclosed herein is evaluated by retention of one or more of the following characteristics after the 45° C. heat treatment: clarity (transparency and translucency), homogeneousness, and cohesiveness.

In aspects of this embodiment, a composition is substantially stable at room temperature for, e.g., about 3 months, about 6 months, about 9 months, about 12 months, about 15 months, about 18 months, about 21 months, about 24 months, about 27 months, about 30 months, about 33 months, or about 36 months. In other aspects of this embodiment, a composition is substantially stable at room temperature for, e.g., at least 3 months, at least 6 months, at least 9 months, at least 12 months, at least 15 months, at least 18 months, at least 21 months, at least 24 months, at least 27 months, at least 30 months, at least 33 months, or at least 36 months. In other aspects of this embodiment, a composition is substantially stable at room temperature for, e.g., about 3 months to about 12 months, about 3 months to about 18 months, about 3 months to about 24 months, about 3 months to about 30 months, about 3 months to about 36 months, about 6 months to about 12 months, about 6 months to about 18 months, about 6 months to about 24 months, about 6 months to about 30 months, about 6 months to about 36 months, about 9 months to about 12 months, about 9 months to about 18 months, about 9 months to about 24 months, about 9 months to about 30 months, about 9 months to about 36 months, about 12 months to about 18 months, about 12 months to about 24 months, about 12 months to about 30 months, about 12 months to about 36 months, about 18 months to about 24 months, about 18 months to about 30 months, or about 18 months to about 36 months.

A composition disclosed herein is in a gel phase at about a physiological temperature. In aspects of this embodiment, a composition is in its gel phase at, e.g., about 26° C., about 27° C., about 28° C., about 29° C., about 30° C., about 31° C., about 32° C., about 33° C., about 34° C., about 35° C., about 36° C., about 37° C., or about 38° C. In other aspects of this embodiment, a composition is in its gel phase at between, e.g., about 28° C. to about 34° C., about 30° C. to about 34° C., about 32° C. to about 34° C., about 28° C. to about 36° C., about 30° C. to about 36° C., about 32° C. to about 36° C., about 34° C. to about 36° C., about 28° C. to about 38° C., about 30° C. to about 38° C., about 32° C. to about 38° C., or about 34° C. to about 38° C. A thermoresponsive polymer disclosed herein may also have a much lower temperature, such as, e.g., about 16° C. to about 25° C., with the understanding that a composition comprising such a thermoresponsive polymer will have to be chilled to below this temperature in order to shift the polymer into its sol phase. This chilling is necessary in order to facilitate administration of the composition.

Once administered into an individual, a composition disclosed herein will solidify due to the phase transition of the thermoresponisve polymer into its hydrogel state. Once in this gel phase, the resulting theremoresponsive polymer network which makes up the hydrogel will comprise a number of different properties that are not present when the polymer is in its sol phase.

Aspects of the present specification provide, in part, a hydrogel disclosed herein that exhibits a complex modulus, an elastic modulus, a viscous modulus and/or a tan 6. The hydrogels as disclosed herein are viscoelastic in that the hydrogel has an elastic component and a viscous component when a force is applied (stress, deformation). The rheological attribute that described this property is the complex modulus (G*), which defines a composition's total resistance to deformation. The complex modulus is a complex number with a real and imaginary part: G*=G′+iG″. The absolute value of G* is Abs(G*)=Sqrt(G′²+G″²). The complex modulus can be defined as the sum of the elastic modulus (G′) and the viscous modulus (G″). Falcone, et al., Temporary Polysaccharide Dermal Fillers: A Model for Persistence Based on Physical Properties, Dermatol Surg. 35(8): 1238-1243 (2009); Tezel, supra, 2008; Kablik, supra, 2009; Beasley, supra, 2009; each of which is hereby incorporated by reference in its entirety.

Elastic modulus, or modulus of elasticity, refers to the ability of a hydrogel to resists deformation, or, conversely, an object's tendency to be non-permanently deformed when a force is applied to it. Elastic modulus characterizes the firmness of a composition and is also known as the storage modulus because it describes the storage of energy from the motion of the composition. The elastic modulus describes the interaction between elasticity and strength (G′=stress/strain) and, as such, provides a quantitative measurement of a composition's hardness or softness. The elastic modulus of an object is defined as the slope of its stress-strain curve in the elastic deformation region: λ=stress/strain, where λ is the elastic modulus in Pascal's; stress is the force causing the deformation divided by the area to which the force is applied; and strain is the ratio of the change caused by the stress to the original state of the object. Although depending on the speed at which the force is applied, a stiffer composition will have a higher elastic modulus and it will take a greater force to deform the material a given distance, such as, e.g., an injection. Specifying how stresses are to be measured, including directions, allows for many types of elastic moduli to be defined. The three primary elastic moduli are tensile modulus, shear modulus, and bulk modulus.

Viscous modulus is also known as the loss modulus because it describes the energy that is lost as viscous dissipation. Tan δ is the ratio of the viscous modulus and the elastic modulus, tan δ=G″/G′. For tan δ values disclosed in the present specification, a tan δ is obtained from the dynamic modulus at a frequency of 0.628 rad/s. A lower tan δ corresponds to a stiffer, harder, or more elastic composition.

Thus, in an embodiment, a hydrogel disclosed herein exhibits a complex modulus. In aspects of this embodiment, a hydrogel exhibits a complex modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, or about 800 Pa. In other aspects of this embodiment, a hydrogel exhibits a complex modulus of, e.g., at most 25 Pa, at most 50 Pa, at most 75 Pa, at most 100 Pa, at most 125 Pa, at most 150 Pa, at most 175 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, at most 700 Pa, at most 750 Pa, or at most 800 Pa. In yet other aspects of this embodiment, a hydrogel exhibits a complex modulus of, e.g., about 25 Pa to about 150 Pa, about 25 Pa to about 300 Pa, about 25 Pa to about 500 Pa, about 25 Pa to about 800 Pa, about 125 Pa to about 300 Pa, about 125 Pa to about 500 Pa, or about 125 Pa to about 800 Pa.

In another embodiment, a hydrogel disclosed herein exhibits an elastic modulus. In aspects of this embodiment, a hydrogel exhibits an elastic modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1,000 Pa, about 1,200 Pa, about 1,300 Pa, about 1,400 Pa, about 1,500 Pa, about 1,600 Pa, about 1700 Pa, about 1800 Pa, about 1900 Pa, about 2,000 Pa, about 2,100 Pa, about 2,200 Pa, about 2,300 Pa, about 2,400 Pa, or about 2,500 Pa. In other aspects of this embodiment, a hydrogel exhibits an elastic modulus of, e.g., at least 25 Pa, at least 50 Pa, at least 75 Pa, at least 100 Pa, at least 125 Pa, at least 150 Pa, at least 175 Pa, at least 200 Pa, at least 250 Pa, at least 300 Pa, at least 350 Pa, at least 400 Pa, at least 450 Pa, at least 500 Pa, at least 550 Pa, at least 600 Pa, at least 650 Pa, at least 700 Pa, at least 750 Pa, at least 800 Pa, at least 850 Pa, at least 900 Pa, at least 950 Pa, at least 1,000 Pa, at least 1,200 Pa, at least 1,300 Pa, at least 1,400 Pa, at least 1,500 Pa, at least 1,600 Pa, at least 1700 Pa, at least 1800 Pa, at least 1900 Pa, at least 2,000 Pa, at least 2,100 Pa, at least 2,200 Pa, at least 2,300 Pa, at least 2,400 Pa, or at least 2,500 Pa. In yet other aspects of this embodiment, a hydrogel exhibits an elastic modulus of, e.g., at most 25 Pa, at most 50 Pa, at most 75 Pa, at most 100 Pa, at most 125 Pa, at most 150 Pa, at most 175 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, at most 700 Pa, at most 750 Pa, at most 800 Pa, at most 850 Pa, at most 900 Pa, at most 950 Pa, at most 1,000 Pa, at most 1,200 Pa, at most 1,300 Pa, at most 1,400 Pa, at most 1,500 Pa, or at most 1,600 Pa. In still other aspects of this embodiment, a hydrogel exhibits an elastic modulus of, e.g., about 25 Pa to about 150 Pa, about 25 Pa to about 300 Pa, about 25 Pa to about 500 Pa, about 25 Pa to about 800 Pa, about 125 Pa to about 300 Pa, about 125 Pa to about 500 Pa, about 125 Pa to about 800 Pa, about 500 Pa to about 1,600 Pa, about 600 Pa to about 1,600 Pa, about 700 Pa to about 1,600 Pa, about 800 Pa to about 1,600 Pa, about 900 Pa to about 1,600 Pa, about 1,000 Pa to about 1,600 Pa, about 1,100 Pa to about 1,600 Pa, about 1,200 Pa to about 1,600 Pa, about 500 Pa to about 2,500 Pa, about 1,000 Pa to about 2,500 Pa, about 1,500 Pa to about 2,500 Pa, about 2,000 Pa to about 2,500 Pa, about 1,300 Pa to about 1,600 Pa, about 1,400 Pa to about 1,700 Pa, about 1,500 Pa to about 1,800 Pa, about 1,600 Pa to about 1,900 Pa, about 1,700 Pa to about 2,000 Pa, about 1,800 Pa to about 2,100 Pa, about 1,900 Pa to about 2,200 Pa, about 2,000 Pa to about 2,300 Pa, about 2,100 Pa to about 2,400 Pa, or about 2,200 Pa to about 2,500 Pa.

In another embodiment, a hydrogel disclosed herein exhibits a tensile modulus. In aspects of this embodiment, a hydrogel exhibits a tensile modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of this embodiment, a hydrogel exhibits a tensile modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of this embodiment, a hydrogel exhibits a tensile modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In another embodiment, a hydrogel disclosed herein exhibits shear modulus. In aspects of this embodiment, a hydrogel exhibits a shear modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of this embodiment, a hydrogel exhibits a shear modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of this embodiment, a hydrogel exhibits a shear modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In another embodiment, a hydrogel disclosed herein exhibits a bulk modulus. In aspects of this embodiment, a hydrogel exhibits a bulk modulus of, e.g., about 5 GPa, about 6 GPa, about 7 GPa, about 8 GPa, about 9 GPa, about 10 GPa, about 15 GPa, about 20 GPa, about 25 GPa, about 30 GPa, about 35 GPa, about 40 GPa, about 45 GPa, about 50 GPa, about 60 GPa, about 70 GPa, about 80 GPa, about 90 GPa, about 100 GPa. In other aspects of this embodiment, a hydrogel exhibits a bulk modulus of, e.g., at least 5 GPa, at least 6 GPa, at least 7 GPa, at least 8 GPa, at least 9 GPa, at least 10 GPa, at least 15 GPa, at least 20 GPa, at least 25 GPa, at least 30 GPa, at least 35 GPa, at least 40 GPa, at least 45 GPa, at least 50 GPa, at least 60 GPa, at least 70 GPa, at least 80 GPa, at least 90 GPa, at least 100 GPa. In yet other aspects of this embodiment, a hydrogel exhibits a bulk modulus of, e.g., about 5 GPa to about 50 GPa, about 5 GPa to about 100 GPa, about 10 GPa to about 50 GPa, about 10 GPa to about 100 GPa, or about 50 GPa to about 100 GPa.

In another embodiment, a hydrogel disclosed herein exhibits a viscous modulus. In aspects of this embodiment, a hydrogel exhibits a viscous modulus of, e.g., about 10 Pa, about 20 Pa, about 30 Pa, about 40 Pa, about 50 Pa, about 60 Pa, about 70 Pa, about 80 Pa, about 90 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, about 650 Pa, or about 700 Pa. In other aspects of this embodiment, a hydrogel exhibits a viscous modulus of, e.g., at most 10 Pa, at most 20 Pa, at most 30 Pa, at most 40 Pa, at most 50 Pa, at most 60 Pa, at most 70 Pa, at most 80 Pa, at most 90 Pa, at most 100 Pa, at most 150 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, or at most 700 Pa. In yet other aspects of this embodiment, a hydrogel exhibits a viscous modulus of, e.g., about 10 Pa to about 30 Pa, about 10 Pa to about 50 Pa, about 10 Pa to about 100 Pa, about 10 Pa to about 150 Pa, about 70 Pa to about 100 Pa, about 50 Pa to about 350 Pa, about 150 Pa to about 450 Pa, about 250 Pa to about 550 Pa, about 350 Pa to about 700 Pa, about 50 Pa to about 150 Pa, about 100 Pa to about 200 Pa, about 150 Pa to about 250 Pa, about 200 Pa to about 300 Pa, about 250 Pa to about 350 Pa, about 300 Pa to about 400 Pa, about 350 Pa to about 450 Pa, about 400 Pa to about 500 Pa, about 450 Pa to about 550 Pa, about 500 Pa to about 600 Pa, about 550 Pa to about 650 Pa, or about 600 Pa to about 700 Pa.

In another embodiment, a hydrogel disclosed herein exhibits a tan δ. In aspects of this embodiment, a hydrogel exhibits a tan δ of, e.g., about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5. In other aspects of this embodiment, a hydrogel exhibits a tan δ of, e.g., at most 0.1, at most 0.2, at most 0.3, at most 0.4, at most 0.5, at most 0.6, at most 0.7, at most 0.8, at most 0.9, at most 1.0, at most 1.1, at most 1.2, at most 1.3, at most 1.4, at most 1.5, at most 1.6, at most 1.7, at most 1.8, at most 1.9, at most 2.0, at most 2.1, at most 2.2, at most 2.3, at most 2.4, or at most 2.5. In yet other aspects of this embodiment, a hydrogel exhibits a tan δ of, e.g., about 0.1 to about 0.3, about 0.3 to about 0.5, about 0.5 to about 0.8, about 1.1 to about 1.4, about 1.4 to about 1.7, about 0.3 to about 0.6, about 0.1 to about 0.5, about 0.5 to about 0.9, about 0.1 to about 0.6, about 0.1 to about 1.0, about 0.5 to about 1.5, about 1.0 to about 2.0, or about 1.5 to about 2.5.

A hydrogel disclosed herein has a hardness property. Hardness refers to various properties of an object in the solid phase that gives it high resistance to various kinds of shape change when force is applied. Hardness is measured using a durometer and is a unitless value that ranges from zero to 100. The ability or inability of a hydrogel to be easily compressed will affect its suitability for application in different tissue replacement roles, i.e., mechanical compliance as bone, fat, connective tissue. Hardness will also affect the ability of a hydrogel to be effectively comminuted, the reason being that a hard material may be more easily and consistently comminuted. Hardness will also affect extrudability, as a soft material may be more readily able to be slightly compressed during injection to pack with other particles or change shape to pass through a syringe barrel or needle.

In an embodiment, a hydrogel disclosed herein exhibits low hardness. In aspects of this embodiment, a hydrogel exhibits a hardness of, e.g., about 5, about 10, about 15, about 20, about 25, about 30, or about 35. In other aspects of this embodiment, a hydrogel exhibits a hardness of, e.g., at most 5, at most 10, at most 15, at most 20, at most 25, at most 30, or at most 35. In yet other aspects of this embodiment, a hydrogel exhibits a hardness of, e.g., about 5 to about 35, about 10 to about 35, about 15 to about 35, about 20 to about 35, or about 25 to about 35, about 5 to about 40, about 10 to about 40, about 15 to about 40, about 20 to about 40, about 25 to about 40, or about 30 to about 40.

In an embodiment, a hydrogel disclosed herein exhibits medium hardness. In aspects of this embodiment, a hydrogel exhibits a hardness of, e.g., about 40, about 45, about 50, about 55, or about 60. In other aspects of this embodiment, a hydrogel exhibits a hardness of, e.g., at least 40, at least 45, at least 50, at least 55, or at least 60. In yet other aspects of this embodiment, a hydrogel exhibits a hardness of, e.g., at most 40, at most 45, at most 50, at most 55, or at most 60. In still other aspects of this embodiment, a hydrogel exhibits a hardness of, e.g., about 35 to about 60, about 35 to about 55, about 35 to about 50, about 35 to about 45, about 40 to about 60, about 45 to about 60, about 50 to about 60, about 55 to about 60, about 40 to about 65, about 45 to about 65, about 50 to about 65, about 55 to about 65.

In another embodiment, a hydrogel disclosed herein exhibits high hardness. In aspects of this embodiment, a hydrogel exhibits a hardness of, e.g., about 65, about 70, about 75, about 80, about 85, about 90, about 95, or about 100. In other aspects of this embodiment, a hydrogel exhibits a hardness of, e.g., at least 65, at least 70, at least 75, at least 80, at least 85, at least 90, at least 95, or at least 100. In yet other aspects of this embodiment, a hydrogel exhibits a hardness of, e.g., about 65 to about 100, about 70 to about 100, about 75 to about 100, about 80 to about 100, about 85 to about 100, about 90 to about 100, about 65 to about 75, about 65 to about 80, about 65 to about 85, about 65 to about 90, about 65 to about 95, about 60 to about 75, about 60 to about 80, about 60 to about 85, about 60 to about 90, or about 60 to about 95.

In an embodiment, a hydrogel disclosed herein exhibits high resistant to deformation. In aspects of this embodiment, a hydrogel exhibits resistant to deformation of, e.g., about 100%, about 99%, about 98%, about 97%, about 96%, about 95%, about 94%, about 93%, about 92%, about 91%, about 90%, about 89%, about 88%, about 87%, about 86%, or about 85%. In other aspects of this embodiment, a hydrogel exhibits resistant to deformation of, e.g., at least 99%, at least 98%, at least 97%, at least 96%, at least 95%, at least 94%, at least 93%, at least 92%, at least 91%, at least 90%, at least 89%, at least 88%, at least 87%, at least 86%, or at least 85%. In yet other aspects of this embodiment, a hydrogel exhibits resistant to deformation of, e.g., at most 99%, at most 98%, at most 97%, at most 96%, at most 95%, at most 94%, at most 93%, at most 92%, at most 91%, at most 90%, at most 89%, at most 88%, at most 87%, at most 86%, or at most 85%. In still aspects of this embodiment, a hydrogel exhibits resistant to deformation of, e.g., about 85% to about 100%, about 87% to about 100%, about 90% to about 100%, about 93% to about 100%, about 95% to about 100%, or about 97% to about 100%.

A hydrogel disclosed herein exhibits high tensile strength. Tensile strength has three different definitional points of stress maxima. Yield strength refers to the stress at which material strain changes from elastic deformation to plastic deformation, causing it to deform permanently. Ultimate strength refers to the maximum stress a material can withstand when subjected to tension, compression or shearing. It is the maximum stress on the stress-strain curve. Breaking strength refers to the stress coordinate on the stress-strain curve at the point of rupture, or when the material pulls apart.

In another embodiment, a hydrogel disclosed herein exhibits high yield strength relative to other polymer classes. In aspects of this embodiment, a silk fibroin material exhibits a yield strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a hydrogel exhibits a yield strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a hydrogel exhibits a yield strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a hydrogel exhibits a yield strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In another embodiment, a hydrogel disclosed herein exhibits high ultimate strength. In aspects of this embodiment, a hydrogel exhibits an ultimate strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a hydrogel exhibits an ultimate strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a hydrogel exhibits an ultimate strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a hydrogel exhibits an ultimate strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In another embodiment, a hydrogel disclosed herein exhibits high breaking strength. In aspects of this embodiment, a hydrogel exhibits a breaking strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a hydrogel exhibits a breaking strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a hydrogel exhibits a breaking strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a hydrogel exhibits a breaking strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

A hydrogel disclosed herein has a transparency and/or translucency property. Transparency (also called pellucidity or diaphaneity) is the physical property of allowing light to pass through a material, whereas translucency (also called translucence or translucidity) only allows light to pass through diffusely. The opposite property is opacity. Transparent materials are clear, while translucent ones cannot be seen through clearly. The silk fibroin hydrogels disclosed herein may, or may not, exhibit optical properties such as transparency and translucency. In certain cases, e.g., superficial line filling, it would be an advantage to have an opaque hydrogel. In other cases such as development of a lens or a “humor” for filling the eye, it would be an advantage to have a translucent hydrogel. These properties could be modified by affecting the structural distribution of the hydrogel material. Factors used to control a hydrogel's optical properties include, without limitation, polymer concentration, gel crystallinity, and hydrogel homogeneity.

When light encounters a material, it can interact with it in several different ways. These interactions depend on the nature of the light (its wavelength, frequency, energy, etc.) and the nature of the material. Light waves interact with an object by some combination of reflection, and transmittance with refraction. As such, an optically transparent material allows much of the light that falls on it to be transmitted, with little light being reflected. Materials which do not allow the transmission of light are called optically opaque or simply opaque.

In an embodiment, a hydrogel disclosed herein is optically transparent. In aspects of this embodiment, a hydrogel transmits, e.g., about 75% of the light, about 80% of the light, about 85% of the light, about 90% of the light, about 95% of the light, or about 100% of the light. In other aspects of this embodiment, a hydrogel transmits, e.g., at least 75% of the light, at least 80% of the light, at least 85% of the light, at least 90% of the light, or at least 95% of the light. In yet other aspects of this embodiment, a hydrogel transmits, e.g., about 75% to about 100% of the light, about 80% to about 100% of the light, about 85% to about 100% of the light, about 90% to about 100% of the light, or about 95% to about 100% of the light.

In another embodiment, a hydrogel disclosed herein is optically opaque. In aspects of this embodiment, a hydrogel transmits, e.g., about 5% of the light, about 10% of the light, about 15% of the light, about 20% of the light, about 25% of the light, about 30% of the light, about 35% of the light, about 40% of the light, about 45% of the light, about 50% of the light, about 55% of the light, about 60% of the light, about 65% of the light, or about 70% of the light. In other aspects of this embodiment, a hydrogel transmits, e.g., at most 5% of the light, at most 10% of the light, at most 15% of the light, at most 20% of the light, at most 25% of the light, at most 30% of the light, at most 35% of the light, at most 40% of the light, at most 45% of the light, at most 50% of the light, at most 55% of the light, at most 60% of the light, at most 65% of the light, at most 70% of the light, or at most 75% of the light. In other aspects of this embodiment, a hydrogel transmits, e.g., about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 25% to about 35%, about 25% to about 40%, about 25% to about 45%, about 25% to about 50%, about 25% to about 55%, about 25% to about 60%, about 25% to about 65%, about 25% to about 70%, or about 25% to about 75%, of the light.

In an embodiment, a hydrogel disclosed herein is optically translucent. In aspects of this embodiment, a hydrogel diffusely transmits, e.g., about 75% of the light, about 80% of the light, about 85% of the light, about 90% of the light, about 95% of the light, or about 100% of the light. In other aspects of this embodiment, a hydrogel diffusely transmits, e.g., at least 75% of the light, at least 80% of the light, at least 85% of the light, at least 90% of the light, or at least 95% of the light. In yet other aspects of this embodiment, a hydrogel diffusely transmits, e.g., about 75% to about 100% of the light, about 80% to about 100% of the light, about 85% to about 100% of the light, about 90% to about 100% of the light, or about 95% to about 100% of the light.

A hydrogel disclosed herein exhibits a dynamic viscosity. Viscosity is resistance of a fluid to shear or flow caused by either shear stress or tensile stress. Viscosity describes a fluid's internal resistance to flow caused by intermolecular friction exerted when layers of fluids attempt to slide by one another and may be thought of as a measure of fluid friction. The less viscous the fluid, the greater its ease of movement (fluidity).

Viscosity can be defined in two ways; dynamic viscosity (μ, although η is sometimes used) or kinematic viscosity (v). Dynamic viscosity, also known as absolute or complex viscosity, is the tangential force per unit area required to move one horizontal plane with respect to the other at unit velocity when maintained a unit distance apart by the fluid. The SI physical unit of dynamic viscosity is the Pascal-second (Pa·s), which is identical to N·m-2·s. Dynamic viscosity can be expressed as τ=μ dvx/dz, where τ=shearing stress, μ=dynamic viscosity, and dvx/dz is the velocity gradient over time. For example, if a fluid with a viscosity of one Pa·s is placed between two plates, and one plate is pushed sideways with a shear stress of one Pascal, it moves a distance equal to the thickness of the layer between the plates in one second. Dynamic viscosity symbolize by is also used, is measured with various types of rheometers, devices used to measure the way in which a liquid, suspension or slurry flows in response to applied forces.

Kinematic viscosity (v) is the ratio of dynamic viscosity to density, a quantity in which no force is involved and is defined as follows: v=μ/ρ, where ρ is the dynamic viscosity ρ is density with the SI unit of kg/m³. Kinematic viscosity is usually measured by a glass capillary viscometer as has an SI unit of m²/s.

The viscosity of a material is highly temperature dependent and for either dynamic or kinematic viscosity to be meaningful, the reference temperature must be quoted. For the viscosity values disclosed herein, a dynamic viscosity is measured at 1 Pa with a cone/plane geometry 2°/40 cm and a temperature of 20° C. Examples of the dynamic viscosity of various fluids at 20° C. is as follows: water is about 1.0×10⁻³ Pa·s, blood is about 3-4×10⁻³ Pa·s, vegetable oil is about 60-85×10⁻³ Pa·s, motor oil SE 30 is about 0.2 Pa·s, glycerin is about 1.4 Pa·s, maple syrup is about 2-3 Pa·s, honey is about 10 Pa·s, chocolate syrup is about 10-25 Pa·s, peanut butter is about 150-250 Pa·s, lard is about 1,000 Pa·s, vegetable shortening is about 1,200 Pa·s, and tar is about 30,000 Pa·s.

In aspects of this embodiment, a hydrogel disclosed herein exhibits a dynamic viscosity of, e.g., about 10 Pa·s, about 20 Pa·s, about 30 Pa·s, about 40 Pa·s, about 50 Pa·s, about 60 Pa·s, about 70 Pa·s, about 80 Pa·s, about 90 Pa·s, about 100 Pa·s, about 125 Pa·s, about 150 Pa·s, about 175 Pa·s, about 200 Pa·s, about 225 Pa·s, about 250 Pa·s, about 275 Pa·s, about 300 Pa·s, about 400 Pa·s, about 500 Pa·s, about 600 Pa·s, about 700 Pa·s, about 750 Pa·s, about 800 Pa·s, about 900 Pa·s, about 1,000 Pa·s, about 1,100 Pa·s, or about 1,200 Pa·s. In other aspects of this embodiment, a hydrogel disclosed herein exhibits a dynamic viscosity of, e.g., at most 10 Pa·s, at most 20 Pa·s, at most 30 Pa·s, at most 40 Pa·s, at most 50 Pa·s, at most 60 Pa·s, at most 70 Pa·s, at most 80 Pa·s, at most 90 Pa·s, at most 100 Pa·s, at most 125 Pa·s, at most 150 Pa·s, at most 175 Pa·s, at most 200 Pa·s, at most 225 Pa·s, at most 250 Pa·s, at most 275 Pa·s, at most 300 Pa·s, at most 400 Pa·s, at most 500 Pa·s, at most 600 Pa·s, at most 700 Pa·s, at most 750 Pa·s, at most 800 Pa·s, at most 900 Pa·s, or at most 1000 Pa·s. In yet other aspects of this embodiment, a hydrogel disclosed herein exhibits a dynamic viscosity of, e.g., about 10 Pa·s to about 100 Pa·s, about 10 Pa·s to about 150 Pa·s, about 10 Pa·s to about 250 Pa·s, about 50 Pa·s to about 100 Pa·s, about 50 Pa·s to about 150 Pa·s, about 50 Pa·s to about 250 Pa·s, about 100 Pa·s to about 500 Pa·s, about 100 Pa·s to about 750 Pa·s, about 100 Pa·s to about 1,000 Pa·s, about 100 Pa·s to about 1,200 Pa·s, about 300 Pa·s to about 500 Pa·s, about 300 Pa·s to about 750 Pa·s, about 300 Pa·s to about 1,000 Pa·s, or about 300 Pa·s to about 1,200 Pa·s.

A hydrogel disclosed herein exhibits a physiologically-acceptable osmolarity. As used herein, the term “osmolarity” refers to the concentration of osmotically active solutes in solution. As used herein, the term “a physiologically-acceptable osmolarity” refers to an osmolarity in accord with, or characteristic of, the normal functioning of a living organism. As such, administration of a hydrogel as disclosed herein exhibits an osmolarity that has substantially no long term or permanent detrimental effect when administered to a mammal. Osmolarity is expressed in terms of osmoles of osmotically active solute per liter of solvent (Osmol/L or Osm/L). Osmolarity is distinct from molarity because it measures moles of osmotically active solute particles rather than moles of solute. The distinction arises because some compounds can dissociate in solution, whereas others cannot. The osmolarity of a solution can be calculated from the following expression: Osmol/L=ΣφᵢηᵢCᵢ, where φ is the osmotic coefficient, which accounts for the degree of non-ideality of the solution; η is the number of particles (e.g. ions) into which a molecule dissociates; and C is the molar concentration of the solute; and i is the index representing the identity of a particular solute. The osmolarity of a hydrogel disclosed herein can be measured using a conventional method that measures solutions.

In an embodiment, a hydrogel disclosed herein exhibits a physiologically-acceptable osmolarity. In aspects of this embodiment, a hydrogel exhibits an osmolarity of, e.g., about 100 mOsm/L, about 150 mOsm/L, about 200 mOsm/L, about 250 mOsm/L, about 300 mOsm/L, about 350 mOsm/L, about 400 mOsm/L, about 450 mOsm/L, or about 500 mOsm/L. In other aspects of this embodiment, a hydrogel exhibits an osmolarity of, e.g., at least 100 mOsm/L, at least 150 mOsm/L, at least 200 mOsm/L, at least 250 mOsm/L, at least 300 mOsm/L, at least 350 mOsm/L, at least 400 mOsm/L, at least 450 mOsm/L, or at least 500 mOsm/L. In yet other aspects of this embodiment, a hydrogel exhibits an osmolarity of, e.g., at most 100 mOsm/L, at most 150 mOsm/L, at most 200 mOsm/L, at most 250 mOsm/L, at most 300 mOsm/L, at most 350 mOsm/L, at most 400 mOsm/L, at most 450 mOsm/L, or at most 500 mOsm/L. In still other aspects of this embodiment, a hydrogel exhibits an osmolarity of, e.g., about 100 mOsm/L to about 500 mOsm/L, about 200 mOsm/L to about 500 mOsm/L, about 200 mOsm/L to about 400 mOsm/L, about 300 mOsm/L to about 400 mOsm/L, about 270 mOsm/L to about 390 mOsm/L, about 225 mOsm/L to about 350 mOsm/L, about 250 mOsm/L to about 325 mOsm/L, about 275 mOsm/L to about 300 mOsm/L, or about 285 mOsm/L to about 290 mOsm/L.

Aspects of the present specification provide, in part, a hydrogel disclosed herein that exhibits a physiologically-acceptable osmolality. As used herein, the term “osmolality” refers to the concentration of osmotically active solutes per kilo of solvent in the body. As used herein, the term “a physiologically-acceptable osmolality” refers to an osmolality in accord with, or characteristic of, the normal functioning of a living organism. As such, administration of a hydrogel disclosed herein exhibits an osmolality that has substantially no long term or permanent detrimental effect when administered to a mammal. Osmolality is expressed in terms of osmoles of osmotically active solute per kilogram of solvent (osmol/kg or Osm/kg) and is equal to the sum of the molalities of all the solutes present in that solution. The osmolality of a solution can be measured using an osmometer. The most commonly used instrument in modern laboratories is a freezing point depression osmometer. This instruments measure the change in freezing point that occurs in a solution with increasing osmolality (freezing point depression osmometer) or the change in vapor pressure that occurs in a solution with increasing osmolality (vapor pressure depression osmometer).

In an embodiment, a hydrogel disclosed herein exhibits a physiologically-acceptable osmolality. In aspects of this embodiment, a hydrogel exhibits an osmolality of, e.g., about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, or about 500 mOsm/kg. In other aspects of this embodiment, a hydrogel exhibits an osmolality of, e.g., at least 100 mOsm/kg, at least 150 mOsm/kg, at least 200 mOsm/kg, at least 250 mOsm/kg, at least 300 mOsm/kg, at least 350 mOsm/kg, at least 400 mOsm/kg, at least 450 mOsm/kg, or at least 500 mOsm/kg. In yet other aspects of this embodiment, a hydrogel exhibits an osmolality of, e.g., at most 100 mOsm/kg, at most 150 mOsm/kg, at most 200 mOsm/kg, at most 250 mOsm/kg, at most 300 mOsm/kg, at most 350 mOsm/kg, at most 400 mOsm/kg, at most 450 mOsm/kg, or at most 500 mOsm/kg. In still other aspects of this embodiment, a hydrogel exhibits an osmolality of, e.g., about 100 mOsm/kg to about 500 mOsm/kg, about 200 mOsm/kg to about 500 mOsm/kg, about 200 mOsm/kg to about 400 mOsm/kg, about 300 mOsm/kg to about 400 mOsm/kg, about 270 mOsm/kg to about 390 mOsm/kg, about 225 mOsm/kg to about 350 mOsm/kg, about 250 mOsm/kg to about 325 mOsm/kg, about 275 mOsm/kg to about 300 mOsm/kg, or about 285 mOsm/kg to about 290 mOsm/kg.

A hydrogel disclosed herein is biocompatibility because its high water content and soft nature renders the hydrogel similar to the natural extracellular matrix of biological tissue. As used herein, the term “biocompatible” refers to a material's ability to perform its intended function, with a desired degree of incorporation in the host, without eliciting any undesirable local or systemic effects in that host. Hydrogel biocompatibility minimizes tissue irritation and cell adherence thereby reducing the likelihood of inflammation. Furthermore, the water absorbing capacity of a hydrogel facilitates the accommodation of cells or hydrophilic molecules such as protein and peptides within the polymeric network.

A hydrogel disclosed herein is typically resistant to biodegradation upon in situ formation in an individual. As used herein, the term “resistant to biodegradation” is synonymous with “resistant to bioerosion”, “resistant to bioresorption”, “non-biodegradable”, “non-bioerodable” and “non-bioresorbable” and refers to a composition disclosed herein that is not prone to degrading, eroding, resorbing, decomposing, or breaking down to any substantial or significant degree while implanted in an individual. Non-limiting examples of substantially non-biodegradable or resistance to biodegradation include less than 10% degradation of a hydrogel over a time period measured, less than 5% degradation of a hydrogel composition over a time period measured, less than 3% degradation of a hydrogel composition over a time period measured, less than 1% degradation of a hydrogel composition over a time period measured. In an embodiment, a hydrogel disclosed herein is substantially non-biodegradable or resistant to biodegradation upon formation within an individual.

In aspects of this embodiment, a hydrogel is substantially non-biodegradable or resistance to biodegradation for, e.g., about 10 days, about 20 days, about 30 days, about 40 days, about 50 days, about 60 days, about 70 days, about 80 days, or about 90 days after in situ formation. In other aspects of this embodiment, a hydrogel is substantially non-biodegradable or resistant to biodegradation for, e.g., at least 10 days, at least 20 days, at least 30 days, at least 40 days, at least 50 days, at least 60 days, at least 70 days, at least 80 days, or at least 90 days after in situ formation. In yet other aspects of this embodiment, a hydrogel is substantially non-biodegradable or resistant to biodegradation for, e.g., about 10 days to about 30 days, about 20 days to about 50 days, about 40 days to about 60 days, about 50 days to about 80 days, or about 60 days to about 90 days after in situ formation.

In other aspects of this embodiment, a hydrogel is substantially non-biodegradable or resistance to biodegradation for, e.g., about 3 months, about 6 months, about 9 months, about 12 months, about 15 months, about 18 months, about 21 months, about 24 months, about 27 months, about 30 months, about 33 months, or about 36 months after in situ formation. In other aspects of this embodiment, a hydrogel is substantially non-biodegradable or resistance to biodegradation for, e.g., at least 3 months, at least 6 months, at least 9 months, at least 12 months, at least 15 months, at least 18 months, at least 21 months, at least 24 months, at least 27 months, at least 30 months, at least 33 months, or at least 36 months after in situ formation. In other aspects of this embodiment, a hydrogel is substantially non-biodegradable or resistance to biodegradation for, e.g., about 3 months to about 12 months, about 3 months to about 18 months, about 3 months to about 24 months, about 3 months to about 30 months, about 3 months to about 36 months, about 6 months to about 12 months, about 6 months to about 18 months, about 6 months to about 24 months, about 6 months to about 30 months, about 6 months to about 36 months, about 9 months to about 12 months, about 9 months to about 18 months, about 9 months to about 24 months, about 9 months to about 30 months, about 9 months to about 36 months, about 12 months to about 18 months, about 12 months to about 24 months, about 12 months to about 30 months, about 12 months to about 36 months, about 18 months to about 24 months, about 18 months to about 30 months, or about 18 months to about 36 months after in situ formation.

Aspects of the present specification provide, in part, a composition disclosed herein that is a pharmaceutically-acceptable composition. As used herein, the term “pharmaceutically acceptable” means any molecular entity or composition that does not produce an adverse, allergic or other untoward or unwanted reaction when administered to an individual. A pharmaceutically-acceptable composition is useful for medical and veterinary applications. A pharmaceutically-acceptable composition may be administered to an individual alone, or in combination with other supplementary active ingredients, agents, drugs or hormones.

Aspects of the present specification provide, in part, a composition as disclosed herein comprising a pharmacologically acceptable excipient. As used herein, the term “pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition as disclosed herein can include one or more pharmaceutically acceptable excipients that facilitate processing of an active ingredient into pharmaceutically acceptable compositions. Insofar as any pharmacologically acceptable excipient is not incompatible with the active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

It is further envisioned that a composition disclosed herein may optionally include, without limitation, other pharmaceutically acceptable components, including, without limitation, buffers, preservatives, tonicity adjusters, salts, antioxidants, osmolality adjusting agents, emulsifying agents, wetting agents, sweetening or flavoring agents, and the like.

A pharmaceutically acceptable buffer is a buffer that can be used to prepare a composition disclosed herein, provided that the resulting preparation is pharmaceutically acceptable. Non-limiting examples of pharmaceutically acceptable buffers include acetate buffers, borate buffers, citrate buffers, neutral buffered salines, phosphate buffers, and phosphate buffered salines. Any concentration of a pharmaceutically acceptable buffer can be useful in formulating a pharmaceutical composition disclosed herein, with the proviso that a therapeutically effective amount of the active ingredient is recovered using this effective concentration of buffer. Non-limiting examples of concentrations of physiologically-acceptable buffers occur within the range of about 0.1 mM to about 900 mM. The pH of pharmaceutically acceptable buffers may be adjusted, provided that the resulting preparation is pharmaceutically acceptable. It is understood that acids or bases can be used to adjust the pH of a pharmaceutical composition as needed. Any buffered pH level can be useful in formulating a pharmaceutical composition, with the proviso that a therapeutically effective amount of the matrix polymer active ingredient is recovered using this effective pH level. Non-limiting examples of physiologically-acceptable pH occur within the range of about pH 5.0 to about pH 8.5. For example, the pH of a composition disclosed herein can be about 5.0 to about 8.0, or about 6.5 to about 7.5, about 7.0 to about 7.4, or about 7.1 to about 7.3.

Pharmaceutically acceptable antioxidants include, without limitation, sodium metabisulfite, sodium thiosulfate, acetylcysteine, butylated hydroxyanisole and butylated hydroxytoluene. Pharmaceutically acceptable preservatives include, without limitation, benzalkonium chloride, chlorobutanol, thimerosal, phenylmercuric acetate, phenylmercuric nitrate, a stabilized oxy chloro composition, such as, e.g., PURITE® (Allergan, Inc. Irvine, Calif.) and chelants, such as, e.g., DTPA or DTPA-bisamide, calcium DTPA, and CaNaDTPA-bisamide.

Pharmaceutically acceptable tonicity adjustors useful in a composition disclosed herein include, without limitation, salts such as, e.g., sodium chloride and potassium chloride; and glycerin. The composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition disclosed herein. Other non-limiting examples of pharmacologically acceptable components can be found in, e.g., Ansel, supra, (1999); Gennaro, supra, (2000); Hardman, supra, (2001); and Rowe, supra, (2003), each of which is hereby incorporated by reference in its entirety.

Aspects of the present specification provide, in part, a method of treating a soft tissue or skin condition of an individual by administering a composition disclosed herein. As used herein, the term “treating,” refers to reducing or eliminating in an individual a cosmetic or clinical symptom of a soft tissue or skin condition characterized by a soft tissue or skin imperfection, defect, disease, and/or disorder; or delaying or preventing in an individual the onset of a cosmetic or clinical symptom of a condition characterized by a soft tissue or skin imperfection, defect, disease, and/or disorder. For example, the term “treating” can mean reducing a symptom of a soft tissue or skin condition by, e.g., at least 20%, at least 30%, at least 40%, at least 50%, at least 60%, at least 70%, at least 80%, at least 90% or at least 100%. The effectiveness of a composition disclosed herein in treating a soft tissue or skin condition can be determined by observing one or more cosmetic, clinical symptoms, and/or physiological indicators associated with the condition. An improvement in a soft tissue or skin condition can also be indicated by a reduced need for a concurrent therapy. Those of skill in the art will know the appropriate symptoms or indicators associated with specific soft tissue or skin condition and will know how to determine if an individual is a candidate for treatment with a compound or composition disclosed herein.

Aspects of the present specification provide, in part, a method of cosmetically enhancing a soft tissue or skin condition of an individual by administering a composition disclosed herein. As used herein, the term “cosmetically enhancing” refers to reducing or eliminating a soft tissue or skin imperfection or defect in an individual; or delaying or preventing the onset a soft tissue or skin imperfection or defect in an individual. For example, the term “cosmetically enhancing” can mean reducing a soft tissue or skin imperfection or defect of a soft tissue or skin by, e.g., at least 20%, at least 30%, at least 40%, at least 50%, at least 60%, at least 70%, at least 80%, at least 90% or at least 100%. The effectiveness of a composition disclosed herein in cosmetically enhancing a soft tissue or skin can be determined by observing one or more cosmetic characteristics. Those of skill in the art will know the appropriate cosmetic characteristics associated with specific soft tissue or skin imperfection or defect and will know how to determine if an individual is a candidate for cosmetic enhancement with a compound or composition disclosed herein.

A composition is administered to an individual. An individual is typically a human being of any age, gender or race. Typically, any individual who is a candidate for a conventional procedure to treat a soft tissue condition is a candidate for a method disclosed herein. Although a individual experiencing the signs of aging skin is an adult, individuals experiencing premature aging or other soft tissue or skin conditions suitable for treatment can also be treated or cosmetically enhanced with a composition disclosed herein. In addition, the presently disclosed compositions and methods may apply to individuals seeking a small/moderate enlargement, shape change or contour alteration of a body part or region, which may not be technically possible or aesthetically acceptable with existing soft tissue implant technology. Pre-operative evaluation typically includes routine history and physical examination in addition to thorough informed consent disclosing all relevant risks and benefits of the procedure.

A soft tissue or skin condition includes, without limitation, a soft tissue or skin imperfection, defect, disease, and/or disorder. Non-limiting examples of a soft tissue or skin condition include breast imperfection, defect, disease and/or disorder, such as, e.g., a breast augmentation, a breast reconstruction, mastopexy, micromastia, thoracic hypoplasia, Poland's syndrome, defects due to implant complications like capsular contraction and/or rupture; a facial imperfection, defect, disease or disorder, such as, e.g., a facial augmentation, a facial reconstruction, a mesotherapy, Parry-Romberg syndrome, lupus erythematosus profundus, dermal divots, scars, sunken checks, thin lips, nasal imperfections or defects, retro-orbital imperfections or defects, a facial fold, line and/or wrinkle like a glabellar line, a nasolabial line, a perioral line, and/or a marionette line, and/or other contour deformities or imperfections of the face; a neck imperfection, defect, disease or disorder; a skin imperfection, defect, disease and/or disorder; other soft tissue or skin imperfections, defects, diseases and/or disorders, such as, e.g., an augmentation or a reconstruction of the upper arm, lower arm, hand, shoulder, back, torso including abdomen, buttocks, upper leg, lower leg including calves, foot including plantar fat pad, eye, genitals, or other body part, region or area, or a disease or disorder affecting these body parts, regions or areas; urinary incontinence, fecal incontinence, other forms of incontinence; and gastroesophageal reflux disease (GERD). As used herein, the term “mesotherapy” refers to a non-surgical cosmetic treatment technique of the skin involving intra-epidermal, intra-dermal, and/or subcutaneous injection of an agent administered as small multiple droplets into the epidermis, dermo-epidermal junction, and/or the dermis.

The amount of a composition used with any of the methods as disclosed herein will typically be determined based on the alteration and/or improvement desired, the reduction and/or elimination of a soft tissue or skin condition symptom, the degree of cosmetic enhancement desired, the clinical and/or cosmetic effect desired by the individual and/or physician, and the body part or region being treated or cosmetically enhanced. The effectiveness of composition administration may be manifested by one or more of the following clinical and/or cosmetic measures: altered and/or improved soft tissue or skin shape, altered and/or improved soft tissue or skin size, altered and/or improved soft tissue or skin contour, altered and/or improved tissue function, tissue ingrowth support and/or new collagen deposition, sustained engraftment of composition, improved patient satisfaction and/or quality of life, and decreased use of implantable foreign material.

For example, for breast augmentation procedures, effectiveness of the compositions and methods may be manifested by one or more of the following clinical and/or cosmetic measures: increased breast size, altered breast shape, altered breast contour, sustained engraftment, reduction in the risk of capsular contraction, decreased rate of liponecrotic cyst formation, improved patient satisfaction and/or quality of life, and decreased use of breast implant.

As another example, for a facial soft tissue or skin condition, effectiveness of the compositions and methods may be manifested by one or more of the following clinical and/or cosmetic measures: increased size, shape, and/or contour of facial feature like increased size, shape, and/or contour of lip, cheek or eye region; altered size, shape, and/or contour of facial feature like altered size, shape, and/or contour of lip, cheek or eye region shape; reduction or elimination of a wrinkle, fold or line in the skin; resistance to a wrinkle, fold or line in the skin; rehydration of the skin; increased elasticity to the skin; reduction or elimination of skin roughness; increased and/or improved skin tautness; reduction or elimination of stretch lines or marks; increased and/or improved skin tone, shine, brightness and/or radiance; increased and/or improved skin color, reduction or elimination of skin paleness; sustained engraftment of composition; decreased side effects; improved patient satisfaction and/or quality of life.

As yet another example, for urinary incontinence procedures, effectiveness of the compositions and methods for sphincter support may be manifested by one or more of the following clinical measures: decreased frequency of incontinence, sustained engraftment, improved patient satisfaction and/or quality of life, and decreased use of implantable foreign filler.

In aspects of this embodiment, the amount of a composition administered is, e.g., about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, about 150 g, or about 200 g. In other aspects of this embodiment, the amount of a composition administered is, e.g., about 0.01 g to about 0.1 g, about 0.1 g to about 1 g, about 1 g to about 10 g, about 10 g to about 100 g, or about 50 g to about 200 g. In yet other aspects of this embodiment, the amount of a composition administered is, e.g., about 0.01 mL, about 0.05 mL, about 0.1 mL, about 0.5 mL, about 1 mL, about 5 mL, about 10 mL, about 20 mL, about 30 mL, about 40 mL, about 50 mL, about 60 mL, about 70 g, about 80 mL, about 90 mL, about 100 mL, about 150 mL, or about 200 mL. In other aspects of this embodiment, the amount of a composition administered is, e.g., about 0.01 mL to about 0.1 mL, about 0.1 mL to about 1 mL, about 1 mL to about 10 mL, about 10 mL to about 100 mL, or about 50 mL to about 200 mL.

The duration of treatment or cosmetic enhancement will typically be determined based on the cosmetic and/or clinical effect desired by the individual and/or physician and the body part or region being treated or cosmetically enhanced. In aspects of this embodiment, administration of a composition disclosed herein can treat and/or cosmetically enhance a soft tissue or skin condition for, e.g., about 6 months, about 7 months, about 8 months, about 9 months, about 10 months, about 11 months, about 12 months, about 13 months, about 14 months, about 15 months, about 18 months, or about 24 months. In other aspects of this embodiment, administration of a composition disclosed herein can treat and/or cosmetically enhance a soft tissue or skin condition for, e.g., at least 6 months, at least 7 months, at least 8 months, at least 9 months, at least 10 months, at least 11 months, at least 12 months, at least 13 months, at least 14 months, at least 15 months, at least 18 months, or at least 24 months. In yet aspects of this embodiment, administration of a composition disclosed herein can treat and/or cosmetically enhance a soft tissue or skin condition for, e.g., about 6 months to about 12 months, about 6 months to about 15 months, about 6 months to about 18 months, about 6 months to about 21 months, about 6 months to about 24 months, about 9 months to about 12 months, about 9 months to about 15 months, about 9 months to about 18 months, about 9 months to about 21 months, about 6 months to about 24 months, about 12 months to about 15 months, about 12 months to about 18 months, about 12 months to about 21 months, about 12 months to about 24 months, about 15 months to about 18 months, about 15 months to about 21 months, about 15 months to about 24 months, about 18 months to about 21 months, about 18 months to about 24 months, or about 21 months to about 24 months.

Aspects of the present specification provide, in part, administering a composition disclosed herein. As used herein, the term “administering” means any delivery mechanism that provides a composition disclosed herein to an individual that potentially results in a clinically, therapeutically, or cosmetically beneficial result. The actual delivery mechanism used to administer a composition to an individual can be determined by a person of ordinary skill in the art by taking into account factors, including, without limitation, the type of soft tissue or skin condition, the location of the soft tissue or skin condition, the cause of the soft tissue or skin condition, the severity of the soft tissue or skin condition, the degree of relief desired, the duration of relief desired, the particular composition used, the rate of excretion of the particular composition used, the pharmacodynamics of the particular composition used, the nature of the other compounds included in the particular composition used, the particular route of administration, the particular characteristics, history and risk factors of the individual, such as, e.g., age, weight, general health and the like, or any combination thereof. In an aspect of this embodiment, a composition disclosed herein is administered to a skin region of an individual by injection.

The route of administration of a composition to an individual patient will typically be determined based on the cosmetic and/or clinical effect desired by the individual and/or physician and the body part or region being treated. A composition disclosed herein may be administered by any means known to persons of ordinary skill in the art including, without limitation, syringe with needle, a pistol (for example, a hydropneumatic-compression pistol), catheter, or by direct surgical implantation. The composition disclosed herein can be administered into a skin region such as, e.g., a dermal region or a hypodermal region. For example, a composition disclosed herein can be injected utilizing needles with a diameter of about 0.26 mm to about 0.4 mm and a length ranging from about 4 mm to about 14 mm. Alternately, the needles can be 21 to 32 G and have a length of about 4 mm to about 70 mm. Preferably, the needle is a single-use needle. The needle can be combined with a syringe, catheter, and/or a pistol.

In addition, a composition disclosed herein can be administered once, or over a plurality of times. Ultimately, the timing used will follow quality care standards. For example, a composition disclosed herein can be administered once or over several sessions with the sessions spaced apart by a few days, or weeks. For instance, an individual can be administered a composition disclosed herein every 1, 2, 3, 4, 5, 6, or 7 days or every 1, 2, 3, or 4 weeks. The administration a composition disclosed herein to an individual can be on a monthly or bi-monthly basis or administered every 3, 6, 9, or 12 months.

For a breast soft tissue replacement procedure, the route of administration may include axillary, periareolar, and/or inframammary routes. Alternatively or in addition, a composition may be delivered through a transaxillary endoscopic subpectoral approach. For a facial soft tissue replacement procedure, the route of administration can be frontal, temporal, zygomatic, periocular, amdibula, perioral or chin routes. In urinary incontinence procedures, the route of administration may include transurethral or periurethral routes. Alternatively or in addition, administration may be delivered via an antegrade route. The routes discussed herein do not exclude the use of multiple routes to achieve the desired clinical effect.

Aspects of the present specification provide, in part, a skin region. The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells and Merkels cells.

Aspects of the present specification provide, in part, a dermal region. As used herein, the term “dermal region” refers to the region of skin comprising the epidermal-dermal junction and the dermis including the superficial dermis (papillary region) and the deep dermis (reticular region). The dermis is the layer of skin beneath the epidermis that consists of connective tissue and cushions the body from stress and strain. The dermis is tightly connected to the epidermis by a basement membrane. It also harbors many Mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the Stratum basale of the epidermis. The dermis is structurally divided into two areas: a superficial area adjacent to the epidermis, called the papillary region, and a deep thicker area known as the reticular region.

The papillary region is composed of loose areolar connective tissue. It is named for its fingerlike projections called papillae that extend toward the epidermis. The papillae provide the dermis with a “bumpy” surface that interdigitates with the epidermis, strengthening the connection between the two layers of skin. The reticular region lies deep in the papillary region and is usually much thicker. It is composed of dense irregular connective tissue, and receives its name from the dense concentration of collagenous, elastic, and reticular fibers that weave throughout it. These protein fibers give the dermis its properties of strength, extensibility, and elasticity. Also located within the reticular region are the roots of the hair, sebaceous glands, sweat glands, receptors, nails, and blood vessels. Tattoo ink is held in the dermis. Stretch marks from pregnancy are also located in the dermis.

The hypodermis lies below the dermis. Its purpose is to attach the dermal region of the skin to underlying bone and muscle as well as supplying it with blood vessels and nerves. It consists of loose connective tissue and elastin. The main cell types are fibroblasts, macrophages and adipocytes (the hypodermis contains 50% of body fat). Fat serves as padding and insulation for the body.

In an aspect of this embodiment, a composition disclosed herein is administered to a skin region of an individual by injection into a dermal region or a hypodermal region. In aspects of this embodiment, a composition disclosed herein is administered to a dermal region of an individual by injection into, e.g., an epidermal-dermal junction region, a papillary region, a reticular region, or any combination thereof.

Aspects of the present specification disclose, in part, a method of treating a soft tissue or skin condition of an individual, the method comprising the steps of administering a composition disclosed herein to a site of the soft tissue or skin condition of the individual, wherein upon administration the composition undergoes a sol-gel phase transition to form a hydrogel, thereby improving the condition.

In aspects of this embodiment, a soft tissue or skin condition is a breast tissue condition, a facial tissue condition, a neck condition, a skin condition, an upper arm condition, a lower arm condition, a hand condition, a shoulder condition, a back condition, a torso including abdominal condition, a buttock condition, an upper leg condition, a lower leg condition including calf condition, a foot condition including plantar fat pad condition, an eye condition, a genital condition, or a condition effecting another body part, region or area.

In another aspect of this embodiment, a method of treating skin dehydration comprises the step of administering to an individual suffering from skin dehydration a composition disclosed herein, wherein the administration of the composition rehydrates the skin, thereby treating skin dehydration. In another aspect of this embodiment, a method of treating a lack of skin elasticity comprises the step of administering to an individual suffering from a lack of skin elasticity a composition disclosed herein, wherein the administration of the composition increases the elasticity of the skin, thereby treating a lack of skin elasticity. In yet another aspect of this embodiment, a method of treating skin roughness comprises the step of administering to an individual suffering from skin roughness a composition disclosed herein, wherein the administration of the composition decreases skin roughness, thereby treating skin roughness. In still another aspect of this embodiment, a method of treating a lack of skin tautness comprises the step of administering to an individual suffering from a lack of skin tautness a composition disclosed herein, wherein the administration of the composition makes the skin tauter, thereby treating a lack of skin tautness.

In a further aspect of this embodiment, a method of treating a skin stretch line or mark comprises the step of administering to an individual suffering from a skin stretch line or mark a composition disclosed herein, wherein the administration of the composition reduces or eliminates the skin stretch line or mark, thereby treating a skin stretch line or mark. In another aspect of this embodiment, a method of treating skin paleness comprises the step of administering to an individual suffering from skin paleness a composition disclosed herein, wherein the administration of the composition increases skin tone or radiance, thereby treating skin paleness. In another aspect of this embodiment, a method of treating skin wrinkles comprises the step of administering to an individual suffering from skin wrinkles a composition disclosed herein, wherein the administration of the composition reduces or eliminates skin wrinkles, thereby treating skin wrinkles. In yet another aspect of this embodiment, a method of treating skin wrinkles comprises the step of administering to an individual a composition disclosed herein, wherein the administration of the composition makes the skin resistant to skin wrinkles, thereby treating skin wrinkles.

Once administered, the distribution or shape of the composition disclosed herein can be modified by external manipulation. Such manipulation may be performed while the composition is still in its sol phase, i.e., before formation of the hydrogel, while the composition is in its gel phase, i.e., after formation of the hydrogel, or both while the composition is in its sol phase and gel phase. Modifying the distribution and shape of the composition can be manually done using, e.g., hands and/or fingers, a shaping tool, or a mold shaped to the body region being treated.

### EXAMPLES

The following examples illustrate representative embodiments now contemplated, but should not be construed to limit the disclosed compositions, and methods of soft tissue augmentation using such compositions.

### Example 1

### Use of Dermal Filler Composition for Treating Wrinkles

This example illustrates the use of compositions and methods disclosed herein for treating wrinkles.

A 37-year-old woman presents with fine lines around her eyes and deeper wrinkles on the sides of her mouth. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the affected regions once a week for three weeks; about 1.0 mL to about 2.0 mL of composition into the affected check region. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 2

### Use of Dermal Filler Composition for Treating Wrinkles

This example illustrates the use of compositions and methods disclosed herein for treating a wrinkles.

A 59-year-old man presents with wrinkles between his eyebrows and in the nasolabial folds. Pre-operative evaluation of the person includes routine history and physical examination in addition to thorough informed consent disclosing all relevant risks and benefits of the procedure. The physician evaluating the individual determines that he is a candidate for soft tissue treatment using the compositions and methods disclosed herein. A composition as disclosed herein is administered subcutaneously and under superficial musculature of the affected regions once every 3 months; about 1.5 mL to about 3.0 mL of composition into each affected region. After administration, the physician modifies the distribution and shape of the composition with shaping tool until the desired appearance is achieved. After 7 days, the facial regions of the individual are evaluated by the physician and the physician determines that the treatment was successful. Both the man and his physician are satisfied with the results of the procedure because he looked younger. Approximately one month after the procedure, the man indicates that his quality of life has improved.

### Example 3

### Use of Dermal Filler Composition for Treating Wrinkles

This example illustrates the use of compositions and methods disclosed herein for treating wrinkles.

A 35-year-old woman presents with fine lines across her forehead. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the affected regions once a week for two weeks; about 1.0 mL to about 2.0 mL of composition into the affected forehead region. After administration, the physician modifies the distribution and shape of the composition with a forehead mold until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 4

### Use of Dermal Filler Composition for Treating Wrinkles

This example illustrates the use of compositions and methods disclosed herein for treating wrinkles.

A 44-year-old woman presents with uneven texture on her right cheek resulting from a loss of collagen due to aging. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the affected regions once a week for three weeks; about 3.0 mL to about 4.0 mL of composition into the affected check region. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 5

### Use of Dermal Filler Composition for Treating Wrinkles

This example illustrates the use of compositions and methods disclosed herein for treating wrinkles.

A 62-year-old woman presents with wrinkles across her forehead, on the sides of her eyes, and in the nasolabial folds. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the affected regions; about 1.5 mL to about 2.5 mL of composition into each affected region. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 6

### Use of Dermal Filler Composition for Treating a Scar

This example illustrates the use of compositions and methods disclosed herein for treating a scar.

A 35-year-old man presents with a deep scar across his chin. Pre-operative evaluation of the person includes routine history and physical examination in addition to thorough informed consent disclosing all relevant risks and benefits of the procedure. The physician evaluating the individual determines that he is a candidate for soft tissue treatment using the compositions and methods disclosed herein. A composition as disclosed herein is administered subcutaneously and under superficial musculature of the affected regions; about 1.0 mL to about 2.0 mL of composition into the affected region. After administration, the physician modifies the distribution and shape of the composition with a shaping tool until the desired appearance is achieved. After 7 days, the facial regions of the individual are evaluated by the physician and the physician determines that the treatment was successful. Both the man and his physician are satisfied with the results of the procedure because he looked younger. Approximately one month after the procedure, the man indicates that his quality of life has improved.

### Example 7

### Use of Dermal Filler Composition for Treating a Facial Defect of the Cheek

This example illustrates the use of compositions and methods disclosed herein for treating a facial defect of the cheek.

A 28-year-old woman presents with a lean face. She felt her face looked old, sad and bitter because of the less fullness of her check contour. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the checks regions; about 15 mL of composition into the left and right cheeks. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 8

### Use of Dermal Filler Composition for Treating Facial Imperfection of Eyelids

This example illustrates the use of compositions and methods disclosed herein for treating a facial imperfection of the eyelids.

A 37-year-old woman presents with sunken eyes and this appearance made her look old and fierce. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the upper eyelid regions; about 2.5 mL of composition into the left and right eyelid regions. After administration, the physician modifies the distribution and shape of the composition with a shaping tool until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 9

### Use of Dermal Filler Composition for Treating Wrinkles

This example illustrates the use of compositions and methods disclosed herein for treating wrinkles.

A 55-year-old woman presents with wrinkles around the eyes and cheek areas. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously and under superficial musculature of the upper eyelid and cheek regions; about 1.5 mL of composition into the left and right eyelid and cheek regions. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure because she looked younger. Approximately one month after the procedure, the woman indicates that his quality of life has improved.

### Example 10

### Use of Dermal Filler Composition for Treating a Breast Defect

This example illustrates the use of compositions and methods disclosed herein for treating a breast defect.

A 32-year-old woman presents with complaints that the medial portions of her breast implants are visible, which accentuated the “bony” appearance of her sternum. In addition she felt her breast are too far apart. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously over the lateral sternum and medial breast bilaterally, 15 mL on the right and 10 mL on the left. The composition is administered in a tear like fashion to increase the surface area to volume ratio. After administration, the physician modifies the distribution and shape of the composition with his fingers until the desired appearance is achieved. After 7 days, the facial regions of the individual are evaluated by the physician and the physician determines that the treatment was successful. Both the woman and her physician are satisfied with the results of the procedure. Approximately one month after the procedure, the woman indicates that his quality of life has improved.

### Example 11

### Use of Dermal Filler Composition for Breast Augmentation

This example illustrates the use of compositions and methods disclosed herein for breast augmentation.

A 28-year-old woman presents micromastia or breast hypoplasia. Pre-operative evaluation of the person includes routine history and physical examination in addition to thorough informed consent disclosing all relevant risks and benefits of the procedure. The physician evaluating the individual determines that she is a candidate for soft tissue treatment using the compositions and methods disclosed herein. A composition as disclosed herein is administered subcutaneously using axillary, periareolar, and inframammary routes bilaterally, 90 mL on the right and 145 mL on the left. The composition is administered in a tear like fashion to increase the surface area to volume ratio. After administration, the physician modifies the distribution and shape of the composition with a shaping tool until the desired appearance is achieved. After 7 days, the facial regions of the individual are evaluated by the physician and the physician determines that the treatment was successful. Both the woman and her physician are satisfied with the results of the procedure. Approximately one month after the procedure, the woman indicates that his quality of life has improved.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A liquid composition comprising a biocompatible thermoresponsive polymer, wherein the thermoresponsive polymer has a sol-gel phase transition of between about 27° C. to about 35° C., and wherein the liquid composition does not include a therapeutic agent.

2. The composition of claim 1, wherein the biocompatible thermoresponsive polymer is a PNA-based thermoresponsive polymer.

3. The composition of claim 1, wherein the thermoresponsive polymer is a PVCL-based thermoresponsive polymer, a PMVE-based thermoresponsive polymer, a PEOVE-based thermoresponsive polymer, a PNVIBAm-based thermoresponsive polymer, a PNVBAm-based thermoresponsive polymer, or a PVEE-based thermoresponsive polymer.

4. The composition of claim 1, wherein the thermoresponsive polymer is a PEO/PPO-based thermoresponsive polymer.

5. The composition of claim 4, wherein the PEO/PPO-based thermoresponsive polymer is a PEO/PPO-based homopolymer, a PEO/PPO-based copolymer, a PEO/PPO-based block copolymer or a PEO/PPO-based interpenetrating network copolymer.

6. The composition of claim 1, wherein the thermoresponsive polymer is a PEG-based thermoresponsive polymer.

7. The composition of claim 1, wherein the PEG-based thermoresponsive polymer is a PEG-based homopolymer, a PEG-based copolymer, a PEG-based block copolymer or a PEG-based interpenetrating network copolymer.

8. The composition of claim 6, wherein the PEG-based thermoresponsive polymer is a PEG/polyester-based thermoresponsive polymer.

9. The composition of claim 1, wherein the liquid composition further comprises an anesthetic agent.

10. A method of treating a soft tissue or skin condition in an individual in need thereof, the method comprising the step of administering a liquid composition of claim 1 to a site of the soft tissue or skin condition of the individual, wherein upon administration the composition undergoes a sol-gel phase transition to form a hydrogel, thereby improving the condition.

11. The method of claim 10, wherein the method further comprises modifying the distribution or shape of the liquid composition by external manipulation before the composition undergoes the sol-gel phase transition.

12. The method of claim 10, wherein the soft tissue or skin condition is an augmentation, a reconstruction, a disease, a disorder, a defect, or an imperfection of a body part, region or area.

13. The method of claim 10, wherein the soft tissue or skin condition is a facial augmentation, a facial reconstruction, a facial disease, a facial disorder, a facial defect, or a facial imperfection.

14. The method of claim 10, wherein the soft tissue or skin condition is a facial fold, or a wrinkle.

15. The method of claim 14, wherein the wrinkle is a glabellar line, a nasolabial line, a perioral line, or a marionette line.

16. The method of claim 10, wherein the soft tissue or skin condition is Parry-Romberg syndrome or lupus erythematosus profundus.

17. A method of cosmetically enhancing a soft tissue or skin in an individual in need thereof, the method comprising the step of: a) administering a liquid composition of claim 1 to a site of the soft tissue or skin of the individual; b) modifying the distribution or shape of the liquid composition by external manipulation before the composition undergoes the sol-gel phase transition; wherein upon administration the composition undergoes a sol-gel phase transition to form a hydrogel; wherein hydrogel formation improves a cosmetic imperfection or defect associated with the soft tissue or skin, thereby providing cosmetic enhancement of the soft tissue or skin.

18. A method of cosmetically enhancing a breast in an individual, the method comprising the step of administering a liquid composition of claim 1 to a site of the breast of the individual; wherein upon administration the composition undergoes a sol-gel phase transition to form a hydrogel, wherein hydrogel formation improves a cosmetic imperfection or defect associated with the breast, thereby providing cosmetic enhancement of the breast.

19. The method of claim 18, wherein the method further comprises modifying the distribution or shape of the liquid composition by external manipulation before the composition undergoes the sol-gel phase transition.
